(19) United States
(12) Patent Application Publication
(10) Pub. No.: US 2008/0234252 A1
(43) Pub. Date: Sep. 25, 2008

(54) Compounds Useful in Therapy

(75) Inventors: Justin Stephen Bryans, Kent (GB); Patrick Stephen Johnson, Kent (GB); Lee Richard Roberts, Kent (GB); Thomas Ryckmans, Kent (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 11/914,688

(22) PCT Filed: May 8, 2006
(86) PCT No.: PCT/IB06/01442

§ 371 Date: Dec. 20, 2007

Related U.S. Application Data
(60) Provisional application No. 60/682,753, filed on May 18, 2005.

(51) Int. Cl. A61K 31/55; C07D 249/08; A61K 31/4196; C07D 471/02; C07D 487/02; A61P 15/00; A61K 31/519; A61K 31/437; C07D 221/20; C07D 223/16

(52) U.S. Cl. 514/217.01; 548/264.8; 514/383; 546/85; 514/292; 546/17; 514/278; 546/113; 514/300; 540/594; 544/279; 514/258.1

(57) ABSTRACT

Compounds of formula (I),

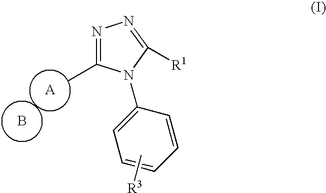

or a pharmaceutically acceptable salt, solvate, ester or amide thereof, wherein
R¹ represents [CH₂]ₙ—R².
   R² represents H, C₁₋₆ alkyloxy or Het;
   n represents a number selected from 0 to 6;
      Het represents an unsaturated heterocycle of 5 or 6 atoms containing one or more heteroatoms selected from O, N, and S;
R³ represents halo;
Ring A represents a 4 to 7 membered, saturated, partially saturated, or unsaturated heterocycle containing one or more heteroatoms selected from O, N, and S;
Ring B represents a saturated, partially saturated, or unsaturated heterocycle of from 3 to 8 atoms containing one or more heteroatoms selected from O, N, and S, or Ring B represents a saturated or unsaturated carbocyclic ring of from 3 to 8 atoms;
Ring B is optionally fused to an aryl ring and is optionally substituted with one or more groups independently selected from R⁴;
Ring A and Ring B share at least one atom;
   R⁴ represents oxo, [CH₂]ₘ—R⁵ or CH—R⁶R⁷;
      R⁵ represents H, OH, C₁₋₆ alkyloxy, COOH, or CONR⁸R⁹;
      m represents a number selected from 0 or 1; and
      R⁶, R⁷, R⁸ and R⁹ independently represent H or C₁₋₆ alkyl;
         are useful for treating a disorder for which a V1a antagonist is indicated, in particular, dysmenorrhoea.

This invention relates to triazole derivatives. To processes for their preparation. To the intermediates used in their preparation. To the compositions containing them, and to the uses of such derivatives and compositions.

The triazole derivatives of the present invention may be useful as vasopressin antagonists. In particular they may be useful as antagonists of the V1a receptor and thus have a number of therapeutic applications, particularly in the treatment of dysmenorrhoea (primary and secondary).

There is a high unmet need in the area of menstrual disorders and it is estimated that up to 90% of all menstruating women are affected to some degree. Up to 42% of women miss work or other activities due to menstrual pain and it has been estimated that around 600 million work hours a year are lost in the US as a result {Coco, A. S. (1999). Primary dysmenorrhoea. [Review] [30 refs]. American Family Physician, 60, 489-96.}.

Menstrual pain in the lower abdomen is caused by myometrial hyperactivity and reduced uterine blood flow. These pathophysiological changes result in abdominal pain that radiates out to the back and legs. This may result in women feeling nauseous, having headaches and suffering from insomnia. This condition is called dysmenorrhoea and can be classified as either primary or secondary dysmenorrhoea.

Primary dysmenorrhoea is diagnosed when no abnormality causing the condition is identified. This affects up to 50% of the female population {Coco, A. S. (1999). Primary dysmenorrhoea. [Review] [30 refs]. American Family Physician, 60, 489-96.; Schroeder, B. & Sanfilippo, J. S. (1999). Dysmenorrhoea and pelvic pain in adolescents. [Review] [78 refs]. Pediatric Clinics of North America, 46, 555-71}. Where an underlying gynaecological disorder is present, such as endometriosis, pelvic inflammatory disease (PID), fibroids or cancers, secondary dysmenorrhoea will be diagnosed. Secondary dysmenorrhoea is diagnosed in only approximately 25% of women suffering from dysmenorrhoea. Dysmenorrhoea can occur in conjunction with menorrhagia, which accounts for around 12% of referrals to gynaecology outpatients departments.

Currently, women suffering from primary dysmenorrhoea are treated with non-steroidal anti-inflammatory drugs (NSAID's) or the oral contraceptive pill. In cases of secondary dysmenorrhoea surgery may be undertaken to correct the underlying gynaecological disorder.

Women suffering from dysmenorrhoea have circulating vasopressin levels which are greater than those observed in healthy women at the same time of the menstrual cycle. Inhibition of the pharmacological actions of vasopressin, at the uterine vasopressin receptor, may prevent dysmenorrhoea.

The compounds of the present invention are therefore potentially useful in the treatment of a wide range of disorders, particularly aggression, Alzheimer's disease, anorexia nervosa, anxiety, anxiety disorder, asthma, atherosclerosis, autism, cardiovascular disease (including angina, atherosclerosis, hypertension, heart failure, edema, hypernatremia), cataract, central nervous system disease, cerebrovascular ischemia, cirrhosis, cognitive disorder, Cushing's disease, depression, diabetes mellitus, dysmenorrhoea (primary and secondary), emesis (including motion sickness), endometriosis, gastrointestinal disease, glaucoma, gynaecological disease, heart disease, intrauterine growth retardation, inflammation (including rheumatoid arthritis), ischemia, ischemic heart disease, lung tumor, micturition disorder, mittlesmerchz, neoplasm, nephrotoxicity, non-insulin dependent diabetes, obesity, obsessive/compulsive disorder, ocular hypertension, preclampsia, premature ejaculation, premature (preterm) labour, pulmonary disease, Raynaud's disease, renal disease, renal failure, male or female sexual dysfunction, septic shock, sleep disorder, spinal cord injury, thrombosis, urogenital tract infection or urolithiasis.

Particularly of interest are the following diseases or disorders:
anxiety, cardiovascular disease (including angina, atherosclerosis, hypertension, heart failure, edema, hypernatremia), dysmenorrhoea (primary and secondary), endometriosis, emesis (including motion sickness), intrauterine growth retardation, inflammation (including rheumatoid arthritis), mittlesmerchz, preclampsia, premature ejaculation, premature (preterm) labour and Raynaud's disease.

The compounds of the invention, and their pharmaceutically acceptable salts and solvates, have the advantage that they are selective inhibitors of the V1a receptor (and so are likely to have reduced side effects), they may have a more rapid onset of action, they may be more potent, they may be longer acting, they may have greater bioavailability or they may have other more desirable properties than the compounds of the prior art.

According to the present invention there is provided a compound of formula (I),

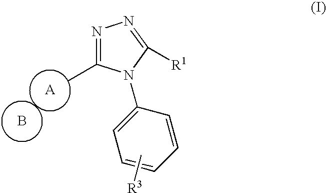

or a pharmaceutically acceptable salt, solvate, ester or amide thereof, wherein:

R¹ represents [CH₂]ₙ—R²;
R² represents H, C₁₋₆ alkyloxy or Het;
n represents a number selected from 0 to 6;
   Het represents an unsaturated heterocycle of 5 or 6 atoms containing one or more heteroatoms selected from O, N, and S;

R³ represents halo;

Ring A represents a 4 to 7 membered, saturated, partially saturated, or unsaturated heterocycle containing one or more heteroatoms selected from O, N, and S;

Ring B represents a saturated, partially saturated, or unsaturated heterocycle of from 3 to 8 atoms containing one or more heteroatoms selected from O, N, and S,

or Ring B represents a saturated or unsaturated carbocyclic ring of from 3 to 8 atoms;

Ring B is optionally fused to an aryl ring and is optionally substituted with one or more groups independently selected from R⁴;

Ring A and Ring B share at least one atom;
R⁴ represents oxo, [CH₂]ₘ—R⁵ or CH—R⁶R⁷;
   R⁵ represents H, OH, C₁₋₆ alkyloxy, COOH, or CONR⁸R⁹;
   m represents a number selected from 0 or 1; and
   R⁶, R⁷, R⁸ and R⁹ independently represent H or C₁₋₆ alkyl.

In the above definitions, halo means fluoro, chloro, bromo or iodo. Alkyl, alkylene and alkyloxy groups, containing the requisite number of carbon atoms, can be unbranched or branched. Examples of alkyl include methyl, ethyl, n-propyl, i-propyl, n-butyl, i-butyl, sec-butyl and t-butyl. Examples of alkyloxy include methoxy, ethoxy, n-propoxy, i-propoxy, n-butoxy, I-butoxy, sec-butoxy and t-butoxy. Examples of alkylene include methylene, 1,1-ethylene, 1,2-ethylene, 1,1-propylene, 1,2-propylene, 1,3-propylene and 2,2-propylene. Het represents a heterocyclic group, examples of which include tetrahydrofuranyl, tetrahyd rothiophenyl, pyrrol id inyl, tetrahyd ropyranyl, tetrahyd rothiopyranyl, piperidinyl, 1,4-dioxanyl, 1,4-oxathianyl, morpholinyl, 1,4-dithianyl, piperazinyl, 1,4-azathianyl, 3,4-dihydro-2H-pyranyl, 5,6-dihydro-2H-pyranyl, 2H-pyranyl, 1,2,3,4-tetrahydropyridinyl, 1,2,5,6-tetrahydropyridinyl, pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl and pyrazinyl.

A preferred compound is one in which n represents 1. A preferred compound is one in which R² represents H. An alternative embodiment is a compound in which R² represents methoxy. An alternative embodiment is a compound in which R² represents Het. A preferred compound is one in which Het represents a triazole ring. A preferred compound is one in which R³ represents chloro. A preferred compound is one in which ring A represents a five-membered ring. An alternative embodiment is a compound in which ring A represents a six-membered ring. An alternative embodiment is a compound in which ring A represents a seven-membered ring. A preferred compound is one in which ring A is saturated. An alternative compound is one in which ring A is partially saturated. A preferred embodiment is a compound in which ring A contains one or two N atoms, particularly preferred is when it contains one N atom. A preferred embodiment is a compound in which ring A represents a piperidinyl ring. An alternative embodiment is a compound in which ring A represents a pyrrolidine ring. An alternative embodiment is a compound in which ring A represents an azepinyl ring. An alternative embodiment is a compound in which ring A represents an imidazolyl ring. An alternative embodiment is a compound in which ring A represents a 2,3-dihydro pyrazinyl ring. A preferred compound is one in which ring B represents a cyclopentyl ring. An alternative embodiment is one in which ring B represents a dihydro-furanyl-2-one ring. An alternative embodiment is one in which ring B represents a furanyl ring. An alternative embodiment is one in which ring B represents a tetrahydrofuranyl ring. An alternative embodiment is one in which ring B represents a pyrrolidinyl-2-one ring. An alternative embodiment is one in which ring B represents a phenyl ring. An alternative embodiment is one in which ring B represents a 2,3-dihydro-1H-pyrrolyl ring. An alternative embodiment is one in which ring B represents a cyclohexa-1,3-dienyl ring. An alternative embodiment is one in which ring B represents a pyrazolyl ring. An alternative embodiment in one in which ring B represents a pyrimidinyl ring. An alternative embodiment is one in which ring B represents an isoxazolyl ring. An alternative embodiment is one in which ring B represents a pyridinyl ring. An alternative embodiment is one in which ring B is fused to a phenyl ring. A preferred embodiment is one in which R⁴ represents methyl, ⁱpropyl, hydroxymethyl, methoxymethyl, CO₂H, CONH₂, CONHMe, or CONMe₂.

The above described embodiments of the invention may be combined with one or more further embodiments such that further embodiments are provided wherein two or more variables are defined more specifically in combination. For example, within the scope of the invention is a further embodiment wherein the variables R¹, R², Ring A, Ring B, and R⁴ all have the more limited definitions assigned to them in the more specific embodiments described above. All such combinations of the more specific embodiments described and defined above are within the scope of the invention

Specific preferred compounds according to the invention are those listed in the Examples section below, and the pharmaceutically acceptable salts, solvates, ester or amide thereof. In particular:
2-[4-(4-Chlorophenyl)-5-(methoxymethyl)-4H-1,2,4-triazol-3-yl]isoindoline;
2-[4-(4-Chlorophenyl)-5-methyl-4H-1,2,4-triazol-3-yl]isoindoline;
2-[4-(4-Chlorophenyl)-5-methyl-4H-1,2,4-triazol-3-yl]-2,3,4,9-tetrahydro-1H-L-carboline;
1′-[4-(4-Chlorophenyl)-5-methyl-4H-1,2,4-triazol-3-yl]-3H-spiro[2-benzofuran-1,4′-piperidin]-3-one;
1′-[4-(4-chlorophenyl)-5-methyl-4H-1,2,4-triazol-3-yl]spiro[isoindole-1,4′-piperidin]-3(2H)-one;
1′-[4-(4-Chlorophenyl)-5-methyl-4H-1,2,4-triazol-3-yl]-3H-spiro[2-benzofuran-1,4′-piperidine];
1′-[4-(4-Chlorophenyl)-5-methyl-4H-1,2,4-triazol-3-yl]-2-methylspiro[isoindole-1,4′-piperidin]-3(2H)-one;
2-[4-(4-Chlorophenyl)-5-(2H-1,2,3-triazol-2-ylmethyl)-4H-1,2,4-triazol-3-yl]isoindoline;
6-[4-(4-Chlorophenyl)-5-(2H-1,2,3-triazol-2-ylmethyl)-4H-1,2,4-triazol-3-yl]-6,7-dihydro-5H-pyrrolo[3,4-b]pyridine;
{2-[4-(4-Chlorophenyl)-5-(2H-1,2,3-triazol-2-ylmethyl)-4H-1,2,4-triazol-3-yl]-2,3-dihydro-1H-isoindol-5-yl}methanol;
1′-[4-(4-chlorophenyl)-5-methyl-4H-1,2,4-triazol-3-yl]-2,3-dihydrospiro[indene-1,4′-piperidine];
{2-[4-(4-Chlorophenyl)-5-methyl-4H-1,2,4-triazol-3-yl]-2,3-dihydro-1H-isoindol-5-yl}methanol;
2-[4-(4-Chlorophenyl)-5-methyl-4H-1,2,4-triazol-3-yl]-5-(methoxymethyl)isoindoline;
2-[4-(4-Chlorophenyl)-5-(2H-1,2,3-triazol-2-ylmethyl)-4H-1,2,4-triazol-3-yl]isoindoline-5-carboxylic acid;
3-[4-(4-Chlorophenyl)-5-methyl-4H-1,2,4-triazol-3-yl]-2,3,4,5-tetrahydro-1H-3-benzazepine;
2-[4-(4-Chloro-phenyl)-5-[1,2,3]triazol-2-ylmethyl-4H-[1,2,4]triazol-3-yl]-quinoxaline;
2-[4-(4-Chloro-phenyl)-5-[1,2,3]triazol-2-ylmethyl-4 H-[1,2,4]triazol-3-]-[1,6]naphthyridine;
3-[4-(4-Chloro-phenyl)-5-[1,2,3]triazol-2-ylmethyl-4H-[1,2,4]triazol-3-yl]-1H-indazole
5-[4-(4-Chlorophenyl)-5-(2H-1,2,3-triazol-2-ylmethyl)-4H-1,2,4-triazol-3-yl]-1-methyl-1,4,5,6-tetrahydropyrrolo[3,4-c]pyrazole;
6-[4-(4-Chlorophenyl)-5-(2H-1,2,3-triazol-2-ylmethyl)-4H-1,2,4-triazol-3-yl]-6,7-dihydro-5H-pyrrolo[3,4-d]pyrimidine;
7-[4-(4-Chlorophenyl)-5-(2H-1,2,3-triazol-2-ylmethyl)-4H-1,2,4-triazol-3-yl]-5,6,7,8-tetrahydropyrido[3,4-d]pyrimidine;
6-[4-(4-Chlorophenyl)-5-(2H-1,2,3-triazol-2-ylmethyl)-4H-1,2,4-triazol-3-yl]-methyl-6,7-dihydro-5H-pyrrolo[3,4-d]pyrimidine;
7-[4-(4-Chlorophenyl)-5-(2H-1,2,3-triazol-2-ylmethyl)-4H-1,2,4-triazol-3-yl]-methyl-5,6,7,8-tetrahydropyrido[3,4-d]pyrimidine;
5-[4-(4-Chlorophenyl)-5-(2H-1,2,3-triazol-2-ylmethyl)-4H-1,2,4-triazol-3-yl]-5,6-dihydro-4H-pyrrolo[3,4-c]isoxazole;
5-[4-(4-Chlorophenyl)-5-(2H-1,2,3-triazol-2-ylmethyl)-4H-1,2,4-triazol-3-yl]-methyl-4,5,6,7-tetrahydro-2H-pyrazolo[4,3-c]pyridine;
5-[4-(4-Chlorophenyl)-5-(2H-1,2,3-triazol-2-ylmethyl)-4H-1,2,4-triazol-3-yl]-isopropyl-4,5,6,7-tetrahydro-2H-pyrazolo[4,3-c]pyridine;
1-[4-(4-Chloro-phenyl)-5-methyl-4H-[1,2,4]triazol-3-yl]-2,3-dihydro-1H-indole-6-carboxylic acid amide;
1-[4-(4-Chloro-phenyl)-5-methyl-4H-[1,2,4]triazol-3-yl]-2,3-dihydro-1H-indole-6-carboxylic acid dimethylamide;
1-[4-(4-Chloro-phenyl)-5-[1,2,3]triazol-2-ylmethyl-4H-1,2,4]triazol-3-dihydro-1H-indole-6-carboxylic acid amide;
1-[4-(4-Chloro-phenyl)-5-[1,2,3]triazol-2-ylmethyl-4H-[1,2,4]triazol-3-yl]-2,3-dihydro-1H-indole-6-carboxylic acid methylamide;
2-[4-(4-Chlorophenyl)-5-(methoxymethyl)-4H-1,2,4-triazol-3-yl]imidazo[1,2-a]pyridine;
and pharmaceutically acceptable salts, solvates, esters and amides thereof.

The compounds of the invention may have the advantage that they are more potent, have a longer duration of action, have a broader range of activity, are more stable, have fewer side effects or are more selective, or have other more useful properties than the compounds of the prior art.

The pharmaceutically acceptable salts of the compounds of formula (I) include the acid addition and base salts thereof.

Suitable acid addition salts are formed from acids which form non-toxic salts. Examples include the acetate, aspartate, benzoate, besylate, bicarbonate/carbonate, bisulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, D- and L-lactate, malate, maleate, malonate, mesylate, methylsulphate, naphthylate, 2-napsylate, nicotinate, nitrate, orotate, oxalate, palmitate, palmoate, phosphate, hydrogen phosphate, dihydrogen phosphate, saccharate, stearate, succinate, sulphate, D- and L-tartrate, tosylate and trifluoroacetate salts. A particularly suitable salt is the besylate derivative of the compounds of the present invention.

Suitable base salts are formed from bases, which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts see Stahl and Wermuth, Handbook of Pharmaceutical Salts: Properties, Selection and Use, Wiley-VCH, Weinheim, Germany (2002).

A pharmaceutically acceptable salt of a compound of formula (I) may be readily prepared by mixing together solutions of the compound of formula (I) and the desired acid or base, as appropriate. The salt may precipitate from solution and be collected by filtration or may be recovered by evaporation of the solvent. The degree of ionisation in the salt may vary from completely ionised to almost non-ionised.

The compounds of the invention may exist in both unsolvated and solvated forms. The term “solvate” is used herein to describe a molecular complex comprising the compound of the invention and one or more pharmaceutically acceptable solvent molecules, for example, ethanol. The term “hydrate” is employed when said solvent is water.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components what may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

Hereinafter all references to compounds of formula (I) and pharmaceutically acceptable derivatives include references to salts, solvates and complexes thereof and to solvates and complexes of salts thereof.

The compounds of the invention include compounds of formula (I) as hereinbefore defined, polymorphs, prodrugs, and isomers thereof (including optical, geometric and tautomeric isomers) as hereinafter defined and isotopically-labelled compounds of formula (I).

As stated, the invention includes all polymorphs of the compounds of formula (I) as hereinbefore defined.

Also within the scope of the invention are so-called “prodrugs” of the compounds of formula (I). Thus certain derivatives of compounds of formula (I) which may have little or no pharmacological activity themselves can, when administered into or onto the body, be converted into compounds of formula (I) having the desired activity, for example, hydrolytic cleavage. Such derivatives are referred to as “prodrugs”. Further information on the use of prodrugs may be found in “Pro-drugs as Novel Delivery Systems, Vol. 14, ACS Symposium Series (T Higuchi and W Stella) and “Bioreversible Carriers in Drug Design”, Pergamon Press, 1987 (ed. E B Roche, American Pharmaceutical Association).

Prodrugs in accordance with the invention can, for example, be produced by replacing appropriate functionalities present in the compounds of formula (I) with certain moieties know to those skilled in the art as “pro-moieties” as described, for example, in “Design of Prodrugs” by H Bundgaard (Elsevier, 1985). Some examples of prodrugs in accordance with the invention include:
(i) where the compound of formula (I) contains a carboxylic acid functionality (—COOH), an ester thereof, for example, replacement of the hydrogen with C₁₋₈ alkyl;
(ii) where the compound of formula (I) contains an alcohol functionality (—OH), an ether thereof, for example, replacement of the hydrogen with C₁₋₆ alkanoyloxymethyl; and
(iii) where the compound of formula (I) contains a primary or secondary amino functionality (—NH₂ or —NHR where R≠H), an amide thereof, for example, replacement of one or both hydrogens with C₁₋₁₀ alkanoyl.

Further examples of replacement groups in accordance with the foregoing examples and examples of other prodrug types may be found in the aforementioned references.

Finally, certain compounds of formula (I) may themselves act as prodrugs of other compounds of formula (I).

Also within the scope of the invention are the metabolites of the compounds of formula (I) when formed in vivo.

Compounds of formula (I) containing one or more asymmetric carbon atoms can exist as two or more stereoisomers. Where a compound of formula (I) contains an alkenyl or alkenylene group, geometric cis/trans (or Z/E) isomers are possible, and where the compound contains, for example, a keto or oxime group or an aromatic moiety, tautomeric isomerism (‘tautomerism’) may occur. It follows that a single compound may exhibit more than one type of isomerism.

Included within the scope of the present invention are all stereoisomers, geometric isomers and tautomeric forms of the compounds of formula (I), including compounds exhibiting more than one type of isomerism, and mixtures of one or more thereof. Also included are acid addition or base salts wherein the counter ion is optically active, for example, D-lactate or L-lysine, or racemic, for example, DL-tartrate or DL-arginine.

Cis/trans isomers may be separated by conventional techniques well known to those skilled in the art, for example, fractional crystallisation and chromatography.

Conventional techniques for the preparation/isolation of individual enantiomers include chiral synthesis from a suitable optically pure precursor or resolution of the racemate (or the racemate of a salt or derivative) using, for example, chiral HPLC.

Alternatively, the racemate (or racemic precursor) may be reacted with a suitable optically active compound, for example, an alcohol, or, in the case where the compounds of formula (I) contains an acidic or basic moiety, an acid or base such as tartaric acid or 1-phenylethylamine. The resulting diastereomeric mixture may be separated by chromatography and/or fractional crystallisation and one or both of the diastereomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Stereoisomeric conglomerates may be separated by conventional techniques known to those skilled in the art—see, for example, “Stereochemistry of Organic Compounds” by E L Eliel (Wiley, NewYork, 1994).

The present invention also includes all pharmaceutically acceptable isotopic variations of a compound of the formula (I) one or more atoms is replaced by atoms having the same atomic number, but an atomic mass or mass number different from the atomic mass or mass number usually found in nature.

Examples of isotopes suitable for inclusion in the compounds of the invention include isotopes of hydrogen such as ²H and ³H, carbon such as ¹¹C, ¹³C and ¹⁴C, nitrogen such as ¹³N and ¹⁵N, oxygen such as ¹⁵O, ¹⁷O and ¹⁸O, phosphorus such as ³²P, sulphur such as ³⁵S, fluorine such as ¹⁸F, iodine such as ¹²³I and ¹²⁵I, and chlorine such as ³⁶Cl.

Certain isotopically-labelled compounds of formula (I), for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds of formula (I) can generally be prepared by conventional techniques known to those skilled in the art or by processes analogous to those described in the accompanying Examples and Preparations using appropriate isotopically-labelled reagents in place of the non-labelled reagent previously employed.

Pharmaceutically acceptable solvates in accordance with the invention include those wherein the solvent of crystallisation may be isotopically substituted, e.g. D₂O, d₆-acetone and d₆-DMSO.

The compounds of the invention are useful in therapy. Therefore, a further aspect of the invention is the use of a compound of formula (I), or a pharmaceutically salt or solvate thereof, as a medicament.

Without being limited by theory, the compounds of the invention may show activity as V1a antagonists. In particular they may be useful in the treatment of a number of conditions including aggression, Alzheimers disease, anorexia nervosa, anxiety, anxiety disorder, asthma, atherosclerosis, autism, cardiovascular disease (including angina, atherosclerosis, hypertension, heart failure, edema, hypernatremia), cataract, central nervous system disease, cerebrovascular ischemia, cirrhosis, cognitive disorder, Cushing's disease, depression, diabetes mellitus, dysmenorrhoea (primary and secondary), emesis (including motion sickness), endometriosis, gastrointestinal disease, glaucoma, gynaecological disease, heart disease, intrauterine growth retardation, inflammation (including rheumatoid arthritis), ischemia, ischemic heart disease, lung tumor, micturition disorder, mittlesmerchz, neoplasm, nephrotoxicity, non-insulin dependent diabetes, obesity, obsessive/compulsive disorder, ocular hypertension, preclampsia, premature ejaculation, premature (preterm) labor, pulmonary disease, Raynaud's disease, renal disease, renal failure, male or female sexual dysfunction, septic shock, sleep disorder, spinal cord injury, thrombosis, urogenital tract infection or urolithiasis sleep disorder, spinal cord injury, thrombosis, urogenital tract infection, urolithiasis. Particularly of interest is dysmenorrhoea (primary or secondary), more particularly, primary dysmenorrhoea.

Therefore, a further aspect of the invention is the method of treatment of a mammal, including a human being, to treat a disorder for which a V1a antagonist is indicated, comprising administering a therapeutically effective amount of a compound of formula (I), or a pharmaceutically acceptable salt or solvate thereof, to the mammal. In particular, the compounds of formula (I) are useful in treating anxiety, cardiovascular disease (including angina, atherosclerosis, hypertension, heart failure, edema, hypernatremia), dysmenorrhoea (primary and secondary), endometriosis, emesis (including motion sickness), intrauterine growth retardation, inflammation (including rheumatoid arthritis), mittlesmerchz, preclampsia, premature ejaculation, premature (preterm) labour or Raynaud's disease. Even more particularly, they are useful in treating dysmenorrhoea (primary or secondary).

A further aspect of the present invention is the use of a compound of formula (I), or a pharmaceutically acceptable salt or solvate thereof, in the manufacture of a medicament for the treatment of a disorder for which a V1a receptor antagonist is indicated.

All of the compounds of the formula (I) can be prepared by the procedures described in the general methods presented below or by the specific methods described in the Examples section and the Preparations section, or by routine modifications thereof. The present invention also encompasses any or one or more of these processes for preparing the compounds of formula (I), in addition to any novel intermediates used therein.

Unless otherwise provided herein:
WSCDI means 1-(3-dimethylaminopropyl)-3-ethylcarbodiimide hydrochloride;
DCC means N,N′-dicyclohexylcarbodiimide;
HOAT means 1-hydroxy-7-azabenzotriazole;
HOBT means 1-hydroxybenzotriazole hydrate;
PyBOP® means Benzotriazol-1-yloxytris(pyrrolidino)phosphoniumhexa fluorophosphate;
PyBrOP® means bromo-tris-pyrrolidino-phosphoniumhexafluoro phosphate;
HBTU means O-benzotriazol-1-yl-N,N,N′,N′-tetramethyluronium hexafluoro phosphate;
Et₃N means triethylamine;
NMM means N-methylmorpholine;
Boc means tert-butoxycarbonyl;
p-TSA means p-toluenesulphonic acid;
Mel means methyl iodide;
MeTosylate means methyl p-toluenesulphonate;
MeOH means methanol, EtOH means ethanol, n-BuOH means n-butanol; EtOAc means ethyl acetate; MeCN means acetonitrile,
THF means tetrahydrofuran, DMSO means dimethyl sulphoxide, and DCM means dichloromethane, DMF means N,N-dimethylformamide, NMP means N-methyl-2-pyrrolidinone, AcOH means acetic acid, TFA means trifluoroacetic acid;
Me means methyl, Et means ethyl;
Cl means chloro; and
OH means hydroxy.

In the following general methods R¹, R³, R⁴, ring A and B are as previously defined for a compound of the formula (I) unless otherwise stated.

When Ring A is attached to the triazole ring through a N atom, compounds of formula (I) may be prepared as shown in scheme 1 below:

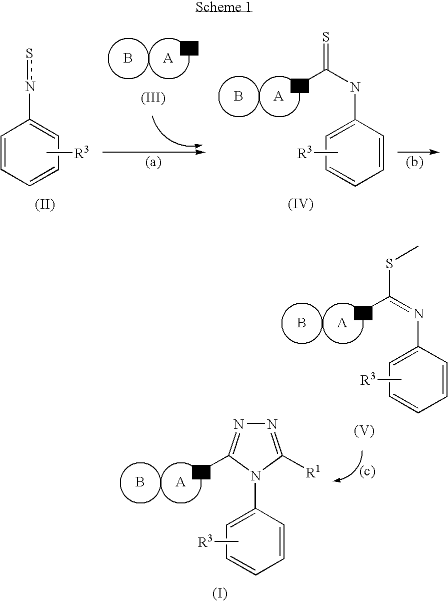

Compounds of formula (II) are available commercially.

Compounds of formula (III) may be prepared from commercial starting materials using standard chemical transformations or by analogy with the methods described in textbooks such as Comprehensive Heterocyclic Chemistry I and II (Pergamon press).

### Step (a): Formation of Thiourea.

The isothiocyanate of formula (II) may be treated with the amine of formula (III), to provide the thiourea of formula (IV). The reaction may be performed in a suitable solvent (e.g. DCM, EtOH), optionally in the presence of a suitable base, such as Et₃N, at about room temperature, for up to 18 hours.

Preferred conditions: 1 eq. amine (III), 0.9 to 1.1eq. isothiocyanate (II) in EtOH, or DCM, at room temperature, for up to 18 hours, optionally in the presence of 1.1 eq. Et₃N.

### Step (b): Alkylation of Thiourea.

Compounds of formula (V) may be prepared by methylation of the thiourea of formula (IV) using a suitable methylating agent (e.g. Mel, MeTosylate) in the presence of a suitable base (e.g. KOt-Bu) in a suitable solvent (e.g. THF, ether) at between 0° C. and the reflux temperature of the solvent, for about 18 hours.

Preferred conditions: 1 eq. (IV), 1.0 to 1.1 eq. KOt-Bu, 1 to 1.1 eq. MeTosylate, in THF at room temperature for up to 18 hours.

### Step (c): Triazole Formation.

Compounds of formula (I) may be prepared by reaction of the compound of formula (V) with a suitable acyl hydrazide (R¹CONHNH₂), optionally under acidic catalysis (e.g. TFA, p-TSA, AcOH), in a suitable solvent (e.g. THF, n-BuOH), at between room temperature and the reflux temperature of the solvent. Preferred conditions: 1 eq. (V), 1.1 to 2.0 eq. of acyl hydrazide (R¹CONHNH₂) in THF at the reflux temperature of the reaction, for up to 18 hours.

Specifically, when R¹ represents CH₃, compounds of formula (I) may be prepared by reaction of the compound of formula (V) with an excess of acetic hydrazide in AcOH, at the reflux temperature of the reaction, for about 18 hours.

Alternatively, the compounds of formula (I) may be prepared as shown in scheme 2 below:

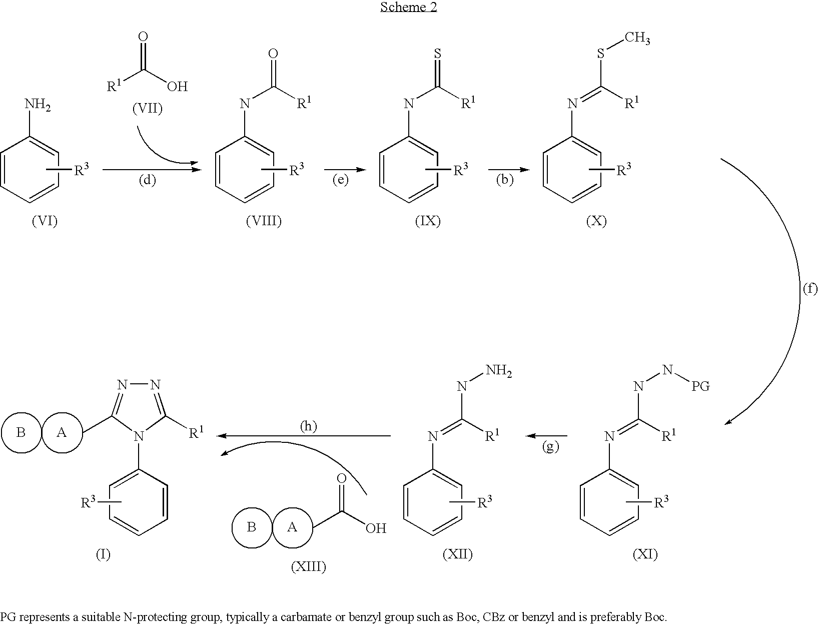

### Step (d):

The compound of formula (VIII) may be prepared by reaction of the acid of formula (VII) with a conventional coupling agent plus amine (VI), optionally in the presence of a catalyst, with an excess of an acid acceptor, in a suitable solvent. Typically the acid (VII) is treated with WSCDI, or DCC, and HOBT, or HOAT, 1 eq. of amine (VI), with an excess of NMM, Et₃N or Hunig's base, in THF, DCM or EtOAc, at room temperature, for between 4 and 48 hours; or an excess of acid (VII) is treated with PYBOP®, or PyBrOP®, or Mukaiyama's reagent, or HBTU, 1 eq. of amine (VI), with an excess of NMM, Et₃N or Hunig's base, in THF, DCM or EtOAc, at room temperature, for between 4 and 24 hours.

Preferred conditions are: 1 eq. amine (VI), 1 eq. acid (VII), 1.1 eq. WSCDI, 3 eq. NMM in DCM, at room temperature, for 18 hours.

The amine of formula (VI) and the acid of formula (VII) are be available commercially, or may be prepared by analogy with literature methods.

### Step (e): Thioamide Formation.

Thionation of the amide (VIII) may be achieved by treatment with a suitable thionating agent (e.g. Lawesson's reagent, P₄S₁₀), optionally in the presence of a base (e.g. Na₂CO₃), in a suitable solvent (e.g. THF), at between 0° C. and the reflux temperature of the solvent.

Preferred conditions are:
4 to 6 eq. P₄S₁₀, 1.5 eq. Na₂CO₃, 1 eq. amide (VIII), in THF, at between 0° C. and reflux, for about 7 days.

The compound of formula (X) may be prepared from the thioamide of formula (IX) according to the method described previously in step (b).

### Step (f):

The compound of formula (X) may be treated with PGNHNH₂ to provide the compound of formula (XI). The reaction may be performed in a suitable solvent (e.g. EtOH, or MeOH), at room temperature, for up to 72 hours.

Preferred conditions: 1 eq. of the compound of formula (X), 1 eq. PGNHNH₂, in EtOH, at room temperature, for up to 72 hours.

### Step (g):

Deprotection of compound (XI) is undertaken using standard methodology, as described in “Protecting Groups in Organic Synthesis” by T. W. Greene and P. Wutz, to provide the compound of formula (XII).

When PG is Boc, the preferred methods are:
hydrogen chloride in a suitable solvent such as methanol or 1,4-dioxane (optionally with methanol as a co-solvent), at room temperature for 1 to 16 hours.

### Step (h):

The compound of formula (I) may be obtained from the compound of formula (XII) by reaction with the acid of formula (XIII), using the methods previously described in step (d).

Preferred conditions are: 1 eq. hydrazine (XII), 1.1 eq. acid (XIII), 1.2 eq. HBTU, 6 eq. Et₃N, in MeCN, at a temperature between room temperature and the reflux temperature of the solvent, for 36 hours.

Alternatively, the compounds of formula (I) may be prepared as shown in scheme 3 below.

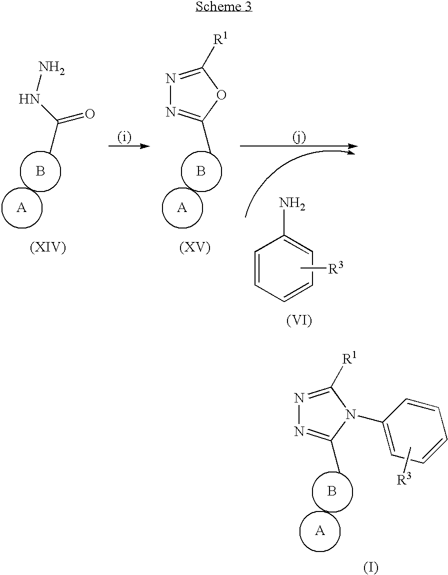

Compounds of formula (XIV) may be obtained as described in WO 9703986 A1, or by reaction of the corresponding lower alkyl ester (e.g. methyl, or ethyl) with hydrazine under standard conditions, as exemplified in the preparations below.

### Step (i):

The compound of formula (XV) may be prepared by reaction of the hydrazide of formula (XIV) with a suitable acetal (e.g. N,N-dimethylacetamide dimethyl acetal), in a suitable solvent, such as THF, or DMF, at between room temperature and about 80° C, for up to 24 hours. The resulting intermediate may then be treated under acid catalysis (e.g. p-TSA, or TFA) in a suitable high boiling solvent (e.g. toluene, or xylene), for up to 3 days, to provide the compound of formula (XV). Preferred conditions: 2.0 eq. of acetal (e.g. 1,1,1,2-tetramethoxy-ethane), in DMF, at between 60° C. and 80° C., for about 23 hours, followed by p-TSA (cat.) in toluene, at reflux for about 3 days.

### Step (j):

Formation of the compound of formula (I) may be achieved by reaction of the compound of formula (XV) with a suitable aniline of formula (VI), in the presence of a suitable acid catalyst, such as TFA or p-TSA, in a suitable high boiling solvent (e.g. toluene or xylene), at an elevated temperature, optionally under microwave radiation.

Preferred conditions: 1 eq. (XV), 0.5 eq. TFA, 1.2 eq. aniline (VI), in toluene, at 170° C., under microwave radiation, for 20 minutes.

Alternatively, compounds of formula (I) may be prepared as shown in scheme 4, below.

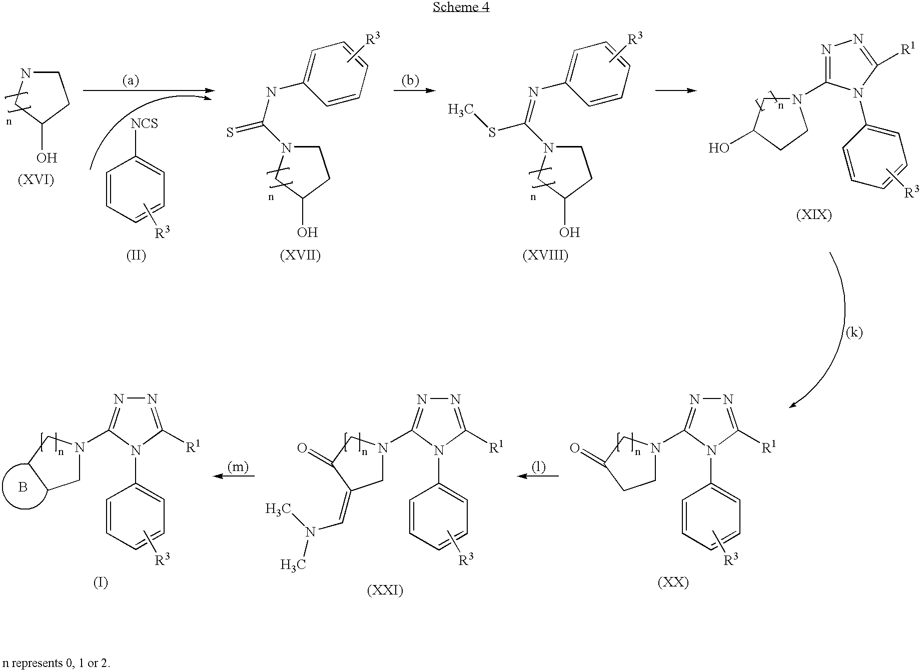

This route has been exemplified in examples 19 to 26, where the A and B rings together represent either
(i) 1,4,5,6-tetrahydropyrrolo[3,4-c]pyrazole;
(ii) 6,7-dihydro-5H-pyrrolo[3,4-d]pyrimidine;
(iii) 5,6,7,8-tetrahydropyrido[3,4-d]pyrimidine;
(iv) 5,6-dihydro-4H-pyrrolo[3,4-c]isoxazole; or
(v) a 4,5,6,7-tetrahydro-2H-pyrazolo[4,3-c]pyridine ring

The compound of formula (XVII) may be prepared from the compounds of formula (XVI) and (II) using the methods previously described in step (a).

The compound of formula (XVIII) may be prepared from the compound from preparation (XVII) using the method previously described in step (b).

The compound of formula (XIX) may be prepared from the compound from preparation (XVIII) using the method previously described in step (c).

### Step (k):

Compounds of formula (XX) may be prepared by oxidation of the compound of formula (XIX) using a suitable mild oxidising agent, or a mixture of agents, in a suitable solvent (e.g. DCM, or DMSO), at low temperature. Typically this may be achieved using the Swern conditions (see Synthesis, 1981, 165), or the Parikh-Doeing reagent (see J. Am. Chem. Soc., 1967, 89, 5505), as described in the literature.

Preferred conditions are: 1.2 eq. (COCl)₂, 2.5 eq. DMSO, at −78° C., for about 30 mins, followed by 1 eq. alcohol (XIX) in DCM, at −78° C., for about 2 hours, followed by 3 eq. Et₃N at between −78° C. and room temperature, for about 72 hours; or 1 eq. alcohol (XIX), 3 eq. sulphur trioxide-pyridine complex, 8 eq. Et₃N, in DMSO, at between 5° C. and room temperature, for about 18 hours.

### Step (I):

The (dimethylamino)ethylidene derivative of formula (XXI) may be prepared by reaction of the compound of formula (XX) with N,N-dimethylacetamide dimethyl acetal in the absence of solvent, at the reflux temperature of the reaction, for up to 4 hours.

Preferred conditions are: 7 eq. N,N-dimethylacetamide dimethyl acetal, 1 eq. compound (XX), at reflux, for between 2 and 4 hours.

### Step (m):

Compounds of formula (I) may be prepared from the compounds of formula (XXI) by standard chemical transformations and by analogy with the methods previously described in the literature (for example the methods of Fukui et al., Heterocycles, 2002, 56, 257; Hojo et al. Synthesis, 1990, 481; Chen et al., J. Het. Chem., 20, 663, 1983; Eiden et al., Arch. Pharm. (Weinheim), 318, 328 to 340, (1985))

When the A and B rings together represent a 1,4,5,6-tetrahydropyrrolo[3,4-c]pyrazole or a 4,5,6,7-tetrahydro-2H-pyrazolo[4,3-c]pyridine ring, the preferred conditions are: 1 eq. compound (XXI), 1.5 to 2.0 eq. R⁴NHNH₂, optionally in the presence of 3 eq. Et₃N, in MeOH, or EtOH, at the reflux temperature of the reaction, for between 3 and 18 hours.

When the A and B ring together represent a 6,7-dihydro-5H-pyrrolo[3,4-d]pyrimidine, or a 5,6,7,8-tetrahydropyrido[3,4-d]pyrimidine ring, the preferred conditions are: 1 eq. compound (XXI), 5 eq. R⁴C(NH)NH₂ (as a salt, typically HCl), 6 eq. Et₃N, in EtOH, at the reflux temperature of the reaction, for 18 hours.

When the A and B ring together represent a 5,6-dihydro-4H-pyrrolo[3,4-c]isoxazole ring the preferred conditions are: 1 eq. compound (XXI), 1.5 eq. NH₂OH.HCl, 2.5 eq. Et₃N, in MeOH, at the reflux temperature of the reaction, for about 18 hours.

The person skilled in the art will appreciate that certain compounds of formula (I) may undergo standard chemical transformations, for example reduction, oxidation, and alkylation reactions, to provide alternative compounds of formula (I). Such transformations are illustrated by the following examples:
(i) Oxidation: see example 14;
(ii) Reduction: see example 6;
(iii) Alkylation: see example 7;
(iv) Amide bond formation: see examples 16 to 18.

Compounds of the invention intended for pharmaceutical use may be administered as crystalline or amorphous products. They may be obtained, for example, as solid plugs, powders, or films by methods such as precipitation, crystallisation, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

A further aspect of the invention is a pharmaceutical formulation including a compound of formula (I), or a pharmaceutically acceptable salt or solvate thereof, together with a pharmaceutically acceptable excipient, diluent or carrier. In a further embodiment there is provided the pharmaceutical formulation for administration either prophylactically or when pain commences.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function.

The compound of formula (I) may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula (I) may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and poly(d/-lactic-coglycolic)acid (PGLA) microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve, which delivers a metered amount. The overall daily dose will typically be in the range 0.01 μg to 15 mg which may be administered in a single dose or, more usually, as divided doses throughout the day.

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to human patients, the total daily dose of the compounds of the invention is typically in the range 0.01 mg to 15 mg depending, of course, on the mode of administration. The total daily dose may be administered in single or divided doses and may, at the physician's discretion, fall outside of the typical range given herein.

These dosages are based on an average human subject having a weight of about 60 kg to 70 kg. The physician will readily be able to determine doses for subjects whose weight falls outside this range, such as infants and the elderly.

For the avoidance of doubt, references herein to “treatment” include references to curative, palliative and prophylactic treatment.

The compounds of the present invention may be tested in the screens set out below:

### 1.0 V_{1A} Filter Binding Assay

### 1.1 Membrane Preparation

Receptor binding assays were performed on cellular membranes prepared from CHO cells stably expressing the human V_{1A} receptor, (CHO-hV_{1A}). The CHO-hV_{1A} cell line was kindly provided under a licensing agreement by Marc Thibonnier, Dept. of Medicine, Case Western Reserve University School of Medicine, Cleveland, Ohio. CHO-hV_{1A} cells were routinely maintained at 37° C. in humidified atmosphere with 5% CO₂ in DMEM/Hams F12 nutrient mix supplemented with 10% fetal bovine serum, 2 mM L-glutamine, 15 mM HEPES and 400 μg/ml G418. For bulk production of cell pellets, adherent CHO-hV_{1A} cells were grown to confluency of 90-100% in 850 cm² roller bottles containing a medium of DMEM/Hams F12 Nutrient Mix supplemented with 10% fetal bovine serum, 2 mM L-glutamine and 15 mM HEPES. Confluent CHO-hV_{1A} cells were washed with phosphate-buffered saline (PBS), harvested into ice cold PBS and centrifuged at 1,000 rpm. Cell pellets were stored at −80° C. until use. Cell pellets were thawed on ice and homogenised in membrane preparation buffer consisting of 50 mM Tris-HCl, pH 7.4, 5 mM MgCl₂ and supplemented with a protease inhibitor cocktail, (Roche). The cell homogenate was centrifuged at 1000 rpm, 10 min, 4° C. and the supernatant was removed and stored on ice. The remaining pellet was homogenised and centrifuged as before. The supernatants were pooled and centrifuged at 25,000×g for 30 min at 4° C. The pellet was resuspended in freezing buffer consisting of 50 mM Tris-HCl, pH 7.4, 5 mM MgCl₂ and 20% glycerol and stored in small aliquots at −80° C. until use. Protein concentration was determined using Bradford reagent and BSA as a standard.

### 1.2 V_{1A} Filter Binding

Protein linearity followed by saturation binding studies were performed on each new batch of membrane. Membrane concentration was chosen that gave specific binding on the linear portion of the curve. Saturation binding studies were then performed using various concentrations of [³H]-arginine vasopressin, [³H]-AVP (0.05 nM-100 nM) and the K_{d} and Bₘₐₓ determined.

Compounds were tested for their effects on [³H]-AVP binding to CHO-hV_{1A} membranes, (³H-AVP; specific activity 65.5 Ci/mmol; NEN Life Sciences). Compounds were solubilised in dimethylsulfoxide (DMSO) and diluted to working concentration of 10% DMSO with assay buffer containing 50 mM Tris-HCL pH 7.4, 5 mM MgCl₂ and 0.05% BSA. 25 μl compound and 25 μl [³H]-AVP, (final concentration at or below K_{d} determined for membrane batch, typically 0.5 nM-0.6 nM) were added to a 96-well round bottom polypropylene plate. The binding reaction was initiated by the addition of 200 μl membrane and the plates were gently shaken for 60 min at room temperature. The reaction was terminated by rapid filtration using a Filtermate Cell Harvester (Packard Instruments) through a 96-well GF/B UniFilter Plate which had been presoaked in 0.5% polyethyleneimine to prevent peptide sticking. The filters were washed three times with 1 ml ice cold wash buffer containing 50 mM Tris-HCL pH 7.4 and 5 mM MgCl₂. The plates were dried and 50 μl Microscint-0 (Packard instruments) was added to each well. The plates were sealed and counted on a TopCount Microplate Scintillation Counter (Packard Instruments). Non-specific binding (NSB) was determined using 1 μM unlabelled d(CH2)5Tyr(Me)AVP ([β-mercapto-β,β-cyclopentamethylenepropionyl, 0-Me-Tyr²,Arg⁸]-vasopressin ) (βMCPVP), (Sigma). The radioligand binding data was analysed using a four parameter logistic equation with the min forced to 0%. The slope was free fitted and fell between −0.75 and −1.25 for valid curves. Specific binding was calculated by subtracting the mean NSB cpm from the mean Total cpm. For test compounds the amount of ligand bound to the receptor was expressed as % bound=(sample cpm−mean NSB cpm)/specific binding cpm×100. The % bound was plotted against the concentration of test compound and a sigmoidal curve was fitted. The inhibitory dissociation constant (Kᵢ) was calculated using the Cheng-Prusoff equation: Kᵢ=IC₅₀/(1+[L]/K_{d}) where [L] is the concentration of ligand present in the well and K_{d} is the dissociation constant of the radioligand obtained from Scatchard plot analysis.

2.0 V_{1A} Functional Assay; Inhibition of AVP/V_{1A}-R mediated Ca²⁺ mobilization by FLIPR (Fluorescent Imaging Plate Reader) (Molecular Devices)

Intracellular calcium release was measured in CHO-hV_{1A} cells using FLIPR, which allows the rapid detection of calcium following receptor activation. The CHO-hV_{1A} cell line was kindly provided under a licensing agreement by Marc Thibonnier, Dept. of Medicine, Case Western Reserve University School of Medicine, Cleveland, Ohio. CHO-V_{1A} cells were routinely maintained at 37° C. in humidified atmosphere with 5% CO₂ in DMEM/Hams F12 nutrient mix supplemented with 10% fetal bovine serum, 2 mM L-glutamine, 15 mM HEPES and 400 μg/ml G418. On the afternoon before the assay cells were plated at a density of 20,000 cells per well into black sterile 96-well plates with clear bottoms to allow cell inspection and fluorescence measurements from the bottom of each well. Wash buffer containing Dulbecco's phosphate buffered saline (DPBS) and 2.5 mM probenecid and loading dye consisting of cell culture medium containing 4 μM Fluo-3-AM (dissolved in DMSO and pluronic acid),(Molecular Probes) and 2.5 mM probenecid was prepared fresh on the day of assay. Compounds were solubilised in DMSO and diluted in assay buffer consisting of DPBS containing 1% DMSO, 0.1% BSA and 2.5 mM probenecid. The cells were incubated with 100 μl loading dye per well for 1 hour at 37° C. in humidified atmosphere with 5% CO₂. After dye loading the cells were washed three times in 100 μl wash buffer using a Denley plate washer. 100 μl wash buffer was left in each well. Intracellular fluorescence was measured using FLIPR. Fluorescence readings were obtained at 2 s intervals with 50 μl of the test compound added after 30 s. An additional 155 measurements at 2 s intervals were then taken to detect any compound agonistic activity. 50 μl of arginine vasopressin (AVP) was then added so that the final assay volume was 200 μl. Further fluorescence readings were collected at 1 s intervals for 120 s. Responses were measured as peak fluorescence intensity (FI). For pharmacological characterization a basal FI was subtracted from each fluorescence response. For AVP dose response curves, each response was expressed as a % of the response to the highest concentration of AVP in that row. For IC₅₀ determinations, each response was expressed as a % of the response to AVP. IC₅₀ values were converted to a modified K_{b} value using the Cheng-Prusoff equation which takes into account the agonist concentration, [A], the agonist EC₅₀ and the slope: K_{b}=IC₅₀/(2+[A]/A₅₀]ⁿ)^{1/n}-1 where [A] is the concentration of AVP, A₅₀ is the EC₅₀ of AVP from the dose response curve and n=slope of the AVP dose response curve.

The compounds of the invention may be administered alone or in combination with one or more other compounds of the invention or in combination with one or more other drugs (or as any combination thereof). The compounds of the present invention may be administered in combination with an oral contraceptive. Thus in a further aspect of the invention, there is provided a pharmaceutical product containing an V1a antagonist and an oral contraceptive as a combined preparation for simultaneous, separate or sequential use in the treatment of dysmenorrhoea.

The compounds of the present invention may be administered in combination with a PDE5 inhibitor. Thus in a further aspect of the invention, there is provided a pharmaceutical product containing a V1a antagonist and a PDEV inhibitor as a combined preparation for simultaneous, separate or sequential use in the treatment of dysmenorrhoea.

PDEV inhibitors useful for combining with V1a antagonists include, but are not limited to:
(i) Preferably 5-[2-ethoxy-5-(4-methyl-1-piperazinylsulphonyl)phenyl]-1-methyl-3-n-propyl-1,6-dihydro-7H-pyrazolo[4,3-d] pyrimidin-7-one (sildenafil, e.g. as sold as Viagra®) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756); 5-(2-ethoxy-5-morpholinoacetylphenyl)-1-methyl-3-n-propyl-1,6-dihydro-7H-pyrazolo[4,3-d]pyrimidin-7-one (see EP-A-0526004); 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166);3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo [4,3-d]pyrimidin-7-one (see WO99/54333); (+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (see WO99/54333); 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2 H-pyrazolo[4,3-d]pyrimidin-5-yl]-3-pyridyl sulphonyl}-4-ethylpiperazine (see WO 01/27113, Example 8);5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15);5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66);5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/ 27112, Example 124); 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132); (6R,12aR)-2,3,6,7,12,12a-hexahydro-2-methyl-6-(3,4-methylenedioxyphenyl)pyrazino[2′,1′:6,1 ]pyrido [3,4-b]indole-1,4-dione (tadalafil, IC-351, Cialis®), i.e. the compound of examples 78 and 95 of published international application WO95/19978, as well as the compound of examples 1, 3, 7 and 8; 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil, LEVITRA®) also known as 1-[[3-(3,4-dihydro-5-methyl-4-oxo-7-propylim idazo[5,1-f]-as-triazin-2-yl)-4-ethoxyphenyl]sulphonyl]-4-ethyl piperazine, i.e. the compound of examples 20, 19, 337 and 336 of published international application WO99/24433;the compound of example 11 of published international application WO93/07124 (EISAI); compounds 3 and 14 from Rotella D P, J. Med. Chem., 2000, 43, 1257; 4-(4-chlorobenzyl)amino-6,7,8-trimethoxyquinazoline; N-[[3-(4,7-dihydro-1-methyl-7-oxo-3-propyl-1H-pyrazolo[4,3-d]-pyrimidin-5-yl)-4-propxyphenyl]sulfonyl]-1-methyl-2-pyrrolidinepropanamide [“DA-8159” (Example 68 of WO00/27848)]; and 7,8-dihydro-8-oxo-6-[2-propoxyphenyl]-1H-imidazo[4,5-g]quinazoline and 1-[3-[1-[(4-fluorophenyl)methyl]-7,8-dihydro-8-oxo-1H-imidazo[4,5-g]quinazolin-6-yl]-4-propoxyphenyl]carboxamide.
(ii) 4-bromo-5-(pyridylmethylamino)-6-[3-(4-chlorophenyl)-propoxy]-3(2H)pyridazinone; 1-[4-[(1,3-benzodioxol-5-ylmethyl)amiono]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl) propoxy)-3-(2H)pyridazinone; I-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer); FR229934 and FR226807 (Fujisawa); and Sch-51866.

The contents of the published patent applications and journal articles and in particular the general formulae of the therapeutically active compounds of the claims and exemplified compounds therein are incorporated herein in their entirety by reference thereto.

Preferably the PDEV inhibitor is selected from sildenafil, tadalafil, vardenafil, DA-8159 and 5-[2-ethoxy-5-(4-ethyl piperazin-1-ylsulphonyl)pyridin-3-yl]-3-ethyl-2-[2-methoxyethyl]-2,6-dihydro-7H-pyrazolo[4,3-d]pyrimidin-7-one.

Most preferably the PDE5 inhibitor is sildenafil and pharmaceutically acceptable salts thereof. Sildenafil citrate is a preferred salt.

The compounds of the present invention may be administered in combination with an NO donor. Thus in a further aspect of the invention, there is provided a pharmaceutical product containing a V1a antagonist and a NO donor as a combined preparation for simultaneous, separate or sequential use in the treatment of dysmenorrhoea.

The compounds of the present invention may be administered in combination with L-arginine, or as an arginate salt. Thus in a further aspect of the invention, there is provided a pharmaceutical product containing a V1a antagonist and L-arginine as a combined preparation for simultaneous, separate or sequential use in the treatment of dysmenorrhoea.

The compounds of the present invention may be administered in combination with a COX inhibitor. Thus in a further aspect of the invention, there is provided a pharmaceutical product containing a V1a antagonist and a COX inhibitor as a combined preparation for simultaneous, separate or sequential use in the treatment of dysmenorrhoea.

COX inhibitors useful for combining with the compounds of the present invention include, but are not limited to:
(i) ibuprofen, naproxen, benoxaprofen, flurbiprofen, fenoprofen, fenbufen, ketoprofen, indoprofen, pirprofen, carprofen, oxaprozin, prapoprofen, miroprofen, tioxaprofen, suprofen, alminoprofen, tiaprofenic acid, fluprofen, bucloxic acid, indomethacin, sulindac, tolmetin, zomepirac, diclofenac, fenclofenec, alclofenac, ibufenac, isoxepac, furofenac, tiopinac, zidometacin, acetyl salicylic acid, indometacin, piroxicam, tenoxicam, nabumetone, ketorolac, azapropazone, mefenamic acid, tolfenamic acid, diflunisal, podophyllotoxin derivatives, acemetacin, droxicam, floctafenine, oxyphenbutazone, phenylbutazone, proglumetacin, acemetacin, fentiazac, clidanac, oxipinac, mefenamic acid, meclofenamic acid, flufenamic acid, niflumic acid, flufenisal, sudoxicam, etodolac, piprofen, salicylic acid, choline magnesium trisalicylate, salicylate, benorylate, fentiazac, clopinac, feprazone, isoxicam and 2-fluoro-a-methyl[1,1′-biphenyl]-4-acetic acid, 4-(nitrooxy)butyl ester (See Wenk, et al., Europ. J. Pharmacol. 453:319-324 (2002));
(ii) meloxicam, (CAS registry number 71125-38-7; described in U.S. Pat. No. 4,233,299), or a pharmaceutically acceptable salt or prodrug thereof;
(iii) celecoxib (U.S. Pat. No. 5,466,823), valdecoxib (U.S. Pat. No. 5,633,272), deracoxib (U.S. Pat. No. 5,521,207), rofecoxib (U.S. Pat. No. 5,474,995), etoricoxib (International Patent Application Publication No. WO 98/03484), JTE-522 (Japanese Patent Application Publication No. 9052882), or a pharmaceutically acceptable salt or prodrug thereof;
(iv) Parecoxib (described in U.S. Pat. No. 5,932,598), which is a therapeutically effective prodrug of the tricyclic Cox-2 selective inhibitor valdecoxib (described in U.S. Pat. No. 5,633,272), in particular sodium parecoxib;
(v) ABT-963 (described in International Patent Application Publication No. WO 00/24719)
(vi) Nimesulide (described in U.S. Pat. No. 3,840,597), flosulide (discussed in J. Carter, Exp. Opin. Ther. Patents, 8(1), 21-29 (1997)), NS-398 (disclosed in U.S. Pat. No. 4,885,367), SD 8381 (described in U.S. Pat. No. 6,034,256), BMS-347070 (described in U.S. Pat. No. 6,180,651), S-2474 (described in European Patent Publication No. 595546) and MK-966 (described in U.S. Pat. No. 5,968,974);

The contents of any of the patent applications, and in particular the general formulae of the therapeutically active compounds of the claims and exemplified compounds therein, are incorporated herein in their entirety by reference thereto.

The following Preparations and Examples illustrate the preparation of compounds of formula (I).

¹H Nuclear magnetic resonance (NMR) spectra were in all cases consistent with the proposed structures. Characteristic chemical shifts (δ) are given in parts-per-million downfield from tetramethylsilane using conventional abbreviations for designation of major peaks: e.g. s, singlet; d, doublet; t, triplet; q, quartet; m, multiplet; br, broad. The mass spectra (m/z) were recorded using either electrospray ionisation (ESI) or atmospheric pressure chemical ionisation (APCI). The following abbreviations have been used for common solvents: CDCl₃, deuterochloroform; D₆-DMSO, deuterodimethylsulphoxide; CD₃OD, deuteromethanol; THF, tetrahydrofuran. “Ammonia” refers to a concentrated solution of ammonia in water possessing a specific gravity of 0.88. Where thin layer chromatography (TLC) has been used it refers to silica gel TLC using silica gel 60 F254 plates, R_{f} is the distance traveled by a compound divided by the distance traveled by the solvent front on a TLC plate. When microwave radiation is employed, the two microwaves used are the Emrys Creator and the Emrys Liberator, both supplied by Personal Chemistry Ltd. The power range is 15-300 W at 2.45 GHz. The actual power supplied varies during the course of the reaction in order to maintain a constant temperature.

Where it is stated that compounds were prepared in the manner described for an earlier Preparation or Example, the skilled person will appreciate that reaction times, number of equivalents of reagents and reaction temperatures may be modified for each specific reaction, and that it may nevertheless be necessary or desirable to employ different work-up or purification conditions.

### Preparation 1: N-(4-Chlorophenyl)-1,3-dihydro-2H-isoindole-2-carbothioamide

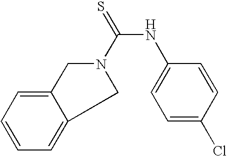

4-Chlorophenyl isothiocyanate (6 g, 35.4 mmol) was added dropwise to a solution of isoindoline (4.8 g, 40.3 mmol) in ethanol (100 ml). The resulting precipitate was filtered off, washed through with ethanol and diethyl ether, and then dried under vacuum to afford the title compound in 97% yield, 9.89 g.

¹HNMR(400 MHz, CDCl₃) δ: 5.01(s, 4H), 7.53-7.42(m, 6H), 7.45(m, 2H), 9.10(s, 1H)

### Preparation 2: N-(4-chlorophenyl)-1,3,4,9-tetrahydro-2H-β-carboline-2-carbothioamide

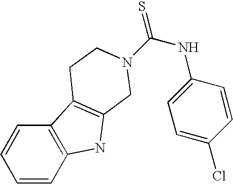

The title compound was prepared from 1,2,3,4-tetrahydro-9H-pyrido[3,4-b]indole and 4-chlorophenyl isothiocyanate, using the same method as that described for preparation 1, in quantitative yield.

¹HNMR(400 MHz, DMSO-d₆) δ: 2.80(m, 2H), 4.17(m, 2H), 5.10(m, 2H), 6.95(m, 1H), 7.02(m, 1H), 7.22-7.32(m, 4H), 7.38(d, 1H), 9.54(s, 1H); LRMS APCl m/z 342 [M+H]⁺

### Preparation 3: N-(4-Chlorophenyl)-3-oxo-2,3-dihydro-1′H-spiro[isoindole-1,4′-piperidine]-1′-carbothioamide

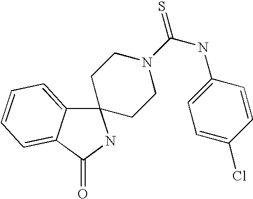

The title compound was prepared from spiro[isoindole-1,4′-piperidin]-3(2H)-one (WO 01/45707, p 83) and 4-chlorophenyl isothiocyanate, using the same method as that described for preparation 1, in 70% yield.

¹HNMR(400 MHz, DMSO-d₆) δ: 1.72-1.86(m, 4H), 4.17-4.29(m, 4H), 6.82(d, 1H), 6.95(m, 1H), 7.18(m, 1H), 7.31(m, 4H), 7.46(d, 1H), 9.41(s, 1H),10.45(s, 1H); LRMS APCI m/z 372 [M+H]⁺

### Preparation 4: N-(4-Chlorophenyl)-1,2,4,5-tetrahydro-3H-3-benzazepine-3-carbothioamide

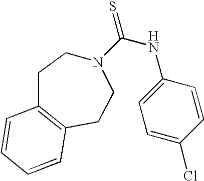

4-Chlorophenyl isothiocyanate (346 mg, 2.04 mmol) was added dropwise to a solution of 1,2,4,5-tetrahydro-3H-3-benzazepine (300 mg, 2.04 mmol) [J. Med. Chem., 2003, 46, 4952] in dichloromethane (20 ml), and the mixture was stirred at room temperature for 1 hour. The reaction mixture was then concentrated in vacuo to afford the title compound as a pale yellow solid in quantitative yield, 660 mg.

¹HNMR(400 MHz, CDCl₃) δ: 3.10(m, 4H), 4.05(m, 4H), 7.08(s, 1H), 7.15-7.31(m, 8H); LRMS APCI m/z 317 [M+H]⁺

### Preparation 5: (3R)-N-(4-chlorophenyl)-3-hydroxypyrrolidine-1-carbothioamide

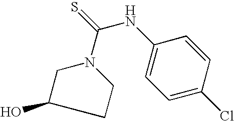

4-Chlorophenyl isothiocyanate (5.83 g, 34 mmol) was added to a solution of (R)-(+)-3-pyrrolidinol (3 g, 34 mmol) in ethanol (12 ml) and the reaction mixture was stirred at room temperature for 15 minutes. The reaction mixture was then concentrated in vacuo and the residue was re-crystallised from dichloromethane to afford the title compound as a solid in 92% yield, 8.2 g.

¹HNMR(400 MHz, CDCl₃) δ: 2.14(m, 2H), 3.80(m, 4H), 4.57(m, 1H), 7.02(s, 1H), 7.31 (m, 4H); LRMS APCI m/z 257 [M+H]⁺

### Preparation 6: N-(4-Chlorophenyl)-4-hydroxypiperidine-1-carbothioamide

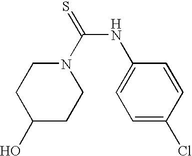

4-Chlorophenyl isothiocyanate (5.02 g, 29.7 mmol) was added to a solution of piperidin-4-ol (3 g, 29.7 mmol) in ethanol (12 ml), and the mixture was stirred at room temperature for 15 minutes. The resulting precipitate was then filtered off and dried under vacuum, for 18 hours at 50° C., to afford the title compound as a solid in 65% yield, 5.2 g.

¹HNMR(400 MHz, CDCl₃) δ: 1.67(m, 2H), 1.96(m, 2H), 3.63(m, 2H), 4.03(m, 1H), 4.10(m, 2H), 7.10(d, 2H), 7.29(d, 2H); LRMS APCI m/z 271 [M+H]⁺

### Preparation 7: N-(4-Chlorophenyl)-3-oxo-1′H,3H-spiro[2-benzofuran-1,4′-piperidine]-1′-carbothioamide

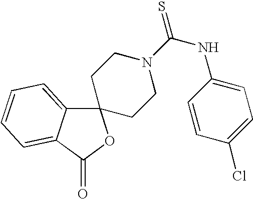

4-Chlorophenyl isothiocyanate (1.3 g, 7.8 mmol) was added dropwise to a solution of 3-oxospiro[isobenzofuran-1(3H),4′-piperidine (1.89 g, 7.8 mmol), [EP 0630887, p 26] in ethanol (20 ml), and the mixture was stirred at room temperature for 18 hours. Further 4-chlorophenyl isothiocyanate (130 mg, 0.78 mmol) was added and the mixture was stirred at room temperature for a further 2 hours. The reaction mixture was then concentrated in vacuo and the residue was triturated with diethyl ether to afford the title compound as a solid in 83% yield, 2.4 g.

¹HNMR(400 MHz, CDCl₃) δ: 1.78(m, 2H), 2.23-2.37(m, 2H), 3.51-3.65(m, 2H), 4.64-4.77(m, 2H), 7.11(d, 2H), 7.31(d, 2H), 7.42(d, 1H), 7.58(m, 1H), 7.71(m, 1H), 7.92(d, 1H); LRMS APCI m/z 373 [M+H]⁺

### Preparation 8: N-(4-Chlorophenyl)-2,3-dihydro-1′H-spiro[indene-1,4′-piperidine]-1′-carbothioamide

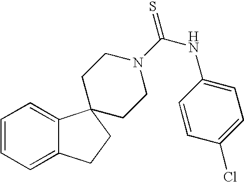

4-Chlorophenyl isothiocyanate (683 mg, 4 mmol) was added dropwise to a solution of 2,3-dihydrospiro[indene-1,4′-piperidine] (900 mg, 4 mmol), [J. Med. Chem. 1992, 35, 2033] and triethylamine (0.67 ml, 4.83 mmol) in dichloromethane (20 ml), and the mixture was then stirred at room temperature for 18 hours. After which time it was partitioned between dichloromethane (200 ml) and an aqueous solution of 0.88 ammonia (100 ml). The aqueous layer was separated and extracted with dichloromethane (2×100 ml) and the combined organic solution was dried over magnesium sulfate and concentrated in vacuo to give a white foam. The foam was triturated with diethyl ether and the residue was dried under vacuum for 18 hours to afford the title compound as a solid in 87% yield, 1.25 g.

¹HNMR(400 MHz, CDCl₃) δ: 1.68(m, 2H), 1.98(m, 2H), 2.12(m, 2H), 2.95(m, 2H), 3.30(t, 2H), 4.60(d, 2H), 7.09-7.35(m, 8H); LRMS ESI m/z 355 [M−H]⁻

### Preparation 9: Methyl N-(4-chlorophenyl)-1,3-dihydro-2H-isoindole-2-carbimidothioate

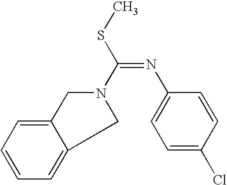

Potassium tert-butoxide (1.35 g, 18.18 mmol) was added to an ice-cold solution of the product of preparation 1 (5 g, 17.3 mmol), in tetrahydrofuran (70 ml), and the mixture was stirred for 10 minutes. Methyl p-toluenesulfonate (3.39 g, 18.18 mmol) was then added and the mixture was stirred for a further 18 hours at room temperature. It was then diluted with ethyl acetate (100 ml) and washed with 2M hydrochloric acid (75 ml), sodium hydrogen carbonate solution (75 ml) and brine (75 ml). The organic solution was then dried over magnesium sulfate, concentrated in vacuo, and the residue was purified by column chromatography on silica gel, eluting with pentane:ethyl acetate, 90:10, to afford the title compound as a white solid in 40% yield, 2.07 g.

¹HNMR(400 MHz, CDCl₃) δ: 2.02(s, 3H), 4.90(brs, 4H), 6.95(m, 2H), 7.20-7.32(m, 6H); LRMS APCI m/z 303 [M+H]⁺

### Preparation 10: Methyl N-(4-chlorophenyl)-1,3,4,9-tetrahydro-2H-L-carboline-2-carbimidothioate

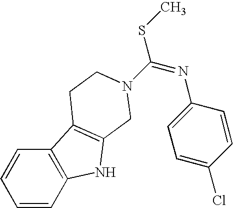

The title compound was prepared from the product of preparation 2 and methyl p-toluenesulfonate, using the same method as that described for preparation 9, as a foam in 38% yield.

### Preparation 11: Methyl N-(4-chlorophenyl)-3-oxo-1′H,3H-spiro[2-benzofuran-1,4′-piperidine]-1′-carbimidothioate

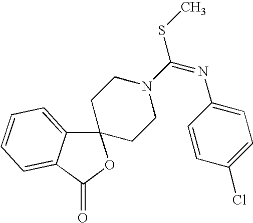

The title compound was prepared from the product of preparation 7 and methyl p-toluenesulfonate, using the same method as that described for preparation 9, as a solid in 77% yield.

¹HNMR(400 MHz, CDCl₃) δ: 1.75(m, 2H), 2.03(s, 3H), 2.23(m, 2H), 3.50(m, 2H), 4.44(m, 2H), 6.89(m, 2H), 7.24(m, 2H), 7.45(m, 1H), 7.55(m, 1H), 7.72(m, 1H), 7.92(m, 1 H); LRMS APCI m/z 387 [M+H]⁺

### Preparation 12: Methyl N-(4-chlorophenyl)-2,3-dihydro-1′H-spiro[indene-1,4′-piperidine]-1′-carbimidothioate

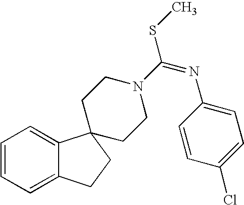

The title compound was prepared from the product of preparation 8 and methyl p-toluenesulfonate, using the same method as that described for preparation 9, as a white solid in 77% yield.

¹HNMR(400 MHz, CDCl₃) δ: 1.63(d, 2H), 1.94(m, 2H), 2.12(m, 5H), 2.95(t, 2H), 3.17(t, 2H), 4.31(d, 2H), 6.88(d, 2H), 7.15-7.27(m, 6H).

### Preparation 13: Methyl N-(4-chlorophenyl)-3-oxo-2,3-dihydro-1′H-spiro[isoindole-1,4′-piperidine]-1′-carbimidothioate

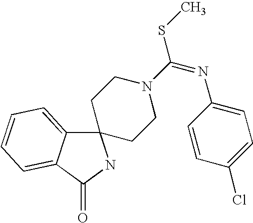

The title compound was prepared from the product of preparation 3 and methyl p-toluenesulfonate, using the same method as that described for preparation 9, as a white foam in 92% yield.

¹HNMR(400 MHz, CDCl₃) δ: 1.84-2.06(m, 4H), 2.10(s, 3H), 3.91-4.03(m, 2H), 4.04-4.17(m, 2H), 6.79(d, 2H), 6.95(m, 1H), 7.14(m, 1H), 7.23(m, 3H), 7.38(m 1H), 8.68(s,1 H); LRMS APCI m/z 386 [M+H]⁺

### Preparation 14: Methyl N-(4-chlorophenyl)-1,2,4,5-tetrahydro-3H-3-benzazepine-3-carbimidothioate

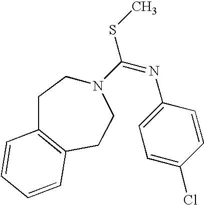

Potassium tert-butoxide (230 mg, 2.05 mmol) was added to an ice-cold solution of the product of preparation 4 (650 mg, 2.05 mmol) in tetrahydrofuran (30 ml) and the mixture was stirred for 10 minutes. Methyl p-toluenesulfonate (382 mg, 2.05 mmol) was then added and the mixture was stirred for a further 2 hours at room temperature. Water (1 ml) was then added and the reaction mixture was concentrated in vacuo. The aqueous residue was partitioned between ethyl acetate (50 ml) and water (30 ml) and the organic layer was separated washed with brine (2×30 ml), dried over magnesium sulfate, and concentrated in vacuo to afford the title compound as a brown oil in quantitative yield, 720 mg.

¹HNMR(400 MHz, CDCl₃) δ: 2.00(s, 3H), 3.00(m, 4H), 3.80-3.93(m, 4H), 6.80-6.88(m, 2H), 7.12-7.22(m, 8H); LRMS APCI m/z 331 [M+H]⁺

### Preparation 15: Methyl (3R)-N-(4-chlorophenyl)-3-hydroxypyrrolidine-1-carbimidothioate

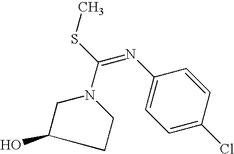

Potassium tert-butoxide (3.59 g, 35 mmol) was added to an ice-cold solution of the product of preparation 5 (8.2 g, 32 mmol) in tetrahydrofuran (40 ml) and the mixture was stirred for 10 minutes. Methyl p-toluenesulfonate (5.96 g, 32 mmol) was then added and the mixture was diluted with further tetrahydrofuran (40 ml) and stirred for a further 1 hour at room temperature. It was then diluted with diethyl ether (100 ml) and washed with water (2×75 ml). The organic solution was dried over magnesium sulfate, and concentrated in vacuo to afford the title compound as an oil in quantitative yield, 9.60 g. ¹HNMR(400 MHz, CDCl₃) δ: 1.85(m, 2H), 1.99(s, 3H), 3.65-3.75(m, 4H), 4.51(m, 1H), 6.96(d, 2H), 7.18(d, 2H); LRMS APCI m/z 271 [M+H]⁺

### Preparation 16: Methyl N-(4-chlorophenyl)-4-hydroxypiperidine-1-carbimidothioate

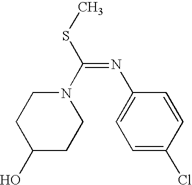

The title compound was prepared form the product of preparation 6 and methyl p-toluenesulfonate, using the same method as that described for preparation 15, as a solid in quantitative yield.

¹HNMR(400 MHz, CDCl₃) δ: 1.59(m, 2H), 1.95(m, 2H), 2.05(s, 3H), 3.25(m, 2H), 3.93(m, 1H), 4.03(m, 2H), 6.82(d, 2H), 7.20(d, 2H); LRMS APCI m/z 285 [M+H]⁺

### Preparation 17: [1,2,3]Triazol-1-yl-acetic acid ethyl ester and [1,2,3]triazol-2-yl-acetic acid ethyl ester

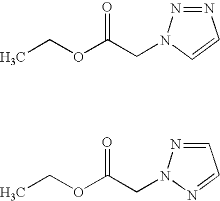

1,2,3-Triazole (19.00 kg, 275 mol) was charged over 30 minutes to a suspension of potassium carbonate (42.15 kg, 305 mol) in ethanol (80 L), and was rinsed in with ethanol (2 L). A solution of ethyl bromoacetate (45.8 kg, 274 mol) in ethanol (30 L) was added slowly and was rinsed in with ethanol (2 L). During this time the reaction temperature was maintained at <20° C. The reaction mixture was then warmed to room temperature and stirred overnight. The suspension was filtered;
washing the residue with ethanol (25 L and 17 L), and then the filtrate was concentrated under reduced pressure. The residue was dissolved in ethyl acetate (120 L) and the solution was washed with 1N hydrochloric acid (1 L 40 L, 7 L 20 L, 4 L 15 L). The aqueous washings were combined and extracted with ethyl acetate (3 L 21 L). The organic phases were combined, dried over magnesium sulfate, and concentrated to in vacuo affording a mixture of the title compounds (25 kg). ¹H NMR spectroscopic analysis indicated that this was a 6:5 mixture of N-2/N-1 isomers.

¹H NMR (400 MHz, CDCl₃): L 1.25 (m, 3H), 4.13 (q, 2H, N-1 isomer), 4.25 (q, 2H, N-2 isomer), 5.20 (s, 2H, N-1 isomer), 5.22 (s, 2H, N-2 isomer), 7.70 (s, 2H, N-2 isomer), 7.77 (s, 2H, N-1 isomer).

### Preparation 18: [1,2,3]Triazol-2-yl-acetic acid hydrazide

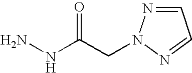

Hydrazine hydrate (8.65 kg, 270 mol) was added to a cooled (<10° C.) solution of the mixture of esters from preparation 17 (19 kg), in ethanol (69 L), keeping the temperature below 20° C. throughout the addition. The reaction mixture was stirred at between 14 and 19° C. for 3 hours, then more ethanol (25 L) was added and the product was collected by filtration, washing with ethanol (10 L). The crude solid was purified by re-crystallisation from ethanol (120 L), followed by three re-crystallisations from methanol (105 L, 120 L and 90 L) to afford the title compound (4.53 kg) after drying in vacuo.

¹H NMR (400 MHz, DMSO-d₆): L 4.33 (s, 2H), 5.02 (s, 2H), 7.77 (s, 2H), 9.40 (s, 1H).

### Preparation 19: 2H-1,2,3-Triazol-2-ylacetic acid

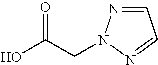

A solution of lithium hydroxide monohydrate (2.5 g, 60 mmol) in water (15 ml) was added to a solution of the product of preparation 17 (7.8 g, 50 ml) and the mixture was stirred at room temperature for 18 hours. It was then acidified with 2M hydrochloric acid (150 ml) and extracted with dichloromethane (150 ml×3). The combined organic solutions were dried over magnesium sulfate and concentrated in vacuo to afford the title compound as a white solid in 81% yield, 5.2 g.

¹HNMR(400 MHz, CDCl₃) δ: 5.35(s, 2H), 7.72(s, 2H); LRMS APCI m/z 128 [M+H]⁺

### Preparation 20: N-(4-Chlorophenyl)-2-(2H-1,2,3-triazol-2-yl)acetamide

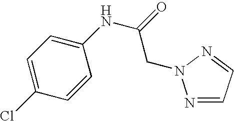

A mixture of the product of preparation 19 (5.2 g, 41 mmol), 4-chloroaniline (5.2 g, 41 mmol), 4-methylmorpholine (12.4 g, 123 mmol), 1-(3-dimethylaminopropyl)-3-ethylcarbodiimide hydrochloride (8.65 g, 45 mmol) and 1-hydroxybenzotriazole hydrate (6.08 g, 45 mmol) in dichloromethane (200 ml) was stirred at room temperature for 18 hours. It was then diluted with dichloromethane (150 ml) and washed with brine (2×100 ml). The organic layer was concentrated in vacuo affording a brown solid. This solid was washed with iso-propyl alcohol (50 ml) to afford a first crop of title compound as a white solid. The aqueous brine washing was then re-extracted with dichloromethane (2×100 ml) and the organic solution was concentrated in vacuo to afford further title compound, providing a total of yield 65%, 6.25 g.

¹HNMR(400 MHz, CDCl₃) δ: 5.24(s, 2H), 7.22(d, 2H), 7.40(d, 2H), 7.78(s, 2H) 8.05(bs, 1 H); LRMS APCI m/z 259 [M+Na]⁺

### Preparation 21: N-(4-Chlorophenyl)-2-(2H-1,2,3-triazol-2-yl)ethanethioamide

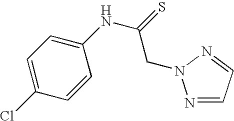

Sodium carbonate (247 mg, 2.33 mmol) was added to a solution of phosphorus pentasulfide (1.04 g, 2.33 mmol) in tetrahydrofuran (80 ml), and the reaction mixture was stirred at room temperature for 1 hour. It was then cooled to 0° C., the product of preparation 20 (1.11 g, 4.67 mmol) was added and the resulting mixture was stirred at 0° C. for 30 minutes, followed by 18 hours at room temperature. It was then heated under reflux for 4.5 hours before further sodium carbonate (494 mg, 4.66 mmol) and phosphorus pentasulfide (1.04 g, 2.33 mmol) were added. Heating was resumed for a further 18 hours, after which time tic analysis indicated that starting material still remained. Further phosphorus pentasulfide (4 g, 8.96 mmol) was added and heating was continued at reflux for 2 hours. The reaction mixture was then re-treated with phosphorus pentasulfide (2 g, 4.48 mmol), heated under reflux for one hour and stirred at room temperature for 5 days. Phosphorus pentasulfide (2 g, 4.48 mmol) was then added and the reaction mixture was heated under reflux for 4 hours, cooled to room temperature and filtered. The filtrate was concentrated in vacuo and the residue was purified by column chromatography on silica gel, eluting with dichloromethane to afford the title compound as a white solid in 81% yield, 951 mg.

¹HNMR(400 MHz, CDCl₃) δ: 5.68(s, 2H), 7.35(d, 2H), 7.63(d, 2H), 7.80(s, 2H) 9.72(s,1H); LRMS APCI m/z 253 [M+H]⁺

### Preparation 22: Methyl-N-(4-chlorophenyl)-2-(2H-1,2,3-triazol-2-yl)ethanimidothioate

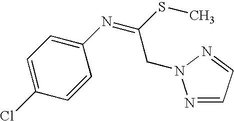

The title compound was prepared form the product of preparation 21 and methyl p-toluenesulfonate, using the same method as that described for preparation 9, a pink gum in quantitative yield.

¹HNMR(400 MHz, CDCl₃) δ: 2.37(s, 3H), 5.22(s, 2H), 7.80(d, 2H), 7.30(m, 2), 7.69(s, 2H); LRMS APCI m/z 267 [M+H]⁺

### Preparation 23: tert-Butyl 2-[N-(4-chlorophenyl)-2-(2H-1,2,3-triazol-2-yl)ethanimidoyl]hydrazinecarboxylate

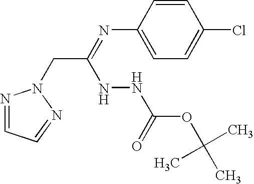

tert-Butyl carbazate (494 mg, 3.74 mmol) was added to a solution of the product of preparation 22 (998 mg, 3.74 mmol) in ethanol (25 ml), and the reaction mixture was stirred at room temperature for 72 hours. It was then diluted with water (200 ml) and extracted with dichloromethane (2×100 ml). The combined organic solutions were washed with brine (2×100 ml), dried over magnesium sulfate and concentrated in vacuo. The residue was triturated with iso-propyl alcohol to afford the title compound as a white solid in 94% yield, 1.23 g.

¹HNMR(400 MHz, CD₃OD) δ: 1.50(s, 9H), 5.30, 5.42(2xs, 2H), 6.80(d, 1H), 7.16(d, 1H), 7.20(d, 1H), 7.52(s, 1H), 7.69(s, 1H), 7.79(s, 1H); LRMS APCI m/z 351 [M+H]⁺

### Preparation 24: N″-(4-chlorophenyl)-2-(2H-1,2,3-triazol-2-yl)ethanimidohydrazide hydrochloride

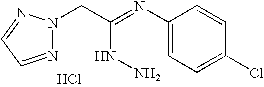

A solution of the product of preparation 23 (1.01 g, 2.88 mmol) in hydrochloric acid (4M in dioxan, 10 ml) was stirred for 18 hours at room temperature. The reaction mixture was then concentrated in vacuo and the residue was azeotroped with methanol to afford the title compound as a white solid in quantitative yield, 945 mg.

¹HNMR(400 MHz, CD₃OD) δ: 5.49(s, 2H), 7.14(d, 2H), 7.39(d, 2H), 7.66(s, 2H); LRMS APCI m/z 251 [M+H]⁺

### Preparation 25: (3R)-1-[4-(4-chlorophenyl)-5-(2H-1,2,3-triazol-2-ylmethyl)-4H-1,2,4-triazol-3-yl]pyrrolidin-3-ol

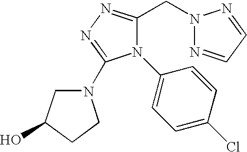

The title compound was prepared from the products of preparations 15 and 18, using the same method as that described for example 1, as a yellow oil in 36% yield.

¹HNMR(400 MHz, CDCl₃) δ: 1.89(m, 2H), 3.00(m, 2H), 3.26(m, 2H), 4.40(m, 1H), 5.49(s, 2H), 7.10(d, 2H), 7.43(d, 2H), 7.49(s, 2H); LRMS APCI m/z 346 [M+H]⁺

### Preparation 26: 1-[4-(4-chlorophenyl)-5-(2H-1,2,3-triazol-2-ylmethyl)-4H-1,2,4-triazol-3-yl]piperidin-4-ol

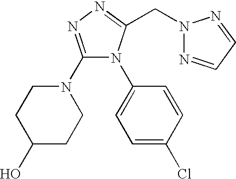

The title compound was prepared from the products of preparations 16 and 18, using the same method as that described for example 1, as a pale orange solid in 73% yield.

¹HNMR(400 MHz, CDCl₃) δ: 1.47(m, 2H), 1.79(m, 2H), 2.92(m, 2H), 3.31(m, 2H), 3.79(m, 1H), 5.58(s, 2H), 7.13(d, 2H), 7.41(d, 2H), 7.51(s, 2H); LRMS APCI m/z 360 [M+H]⁺

### Preparation 27: 1-[4-(4-chlorophenyl)-5-(2H-1,2,3-triazol-2-ylmethyl)-4H-1,2,4-triazol-3-yl]pyrrolidin-3-one

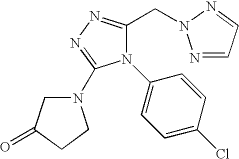

Oxalyl chloride (455 μl, 5.21 mmol) was added to a mixture of dichloromethane (15 ml) and dimethylsulfoxide (769 pl) cooled to −78° C., and the mixture was stirred for 30 minutes. It was then re-cooled to −78° C. and a solution of the product of preparation 25 (1.5 g, 4.34 mmol) in dichloromethane (10 ml) was added dropwise. After stirring for 2 hours, triethylamine (1.81 ml, 13.02 mmol) was added and the mixture was stirred for 72 hours, allowing the temperature to rise to 25° C. It was then diluted with ethyl acetate (100 ml) and washed with saturated sodium hydrogen carbonate solution (50 ml). The organic solution was dried over magnesium sulfate and concentrated in vacuo to afford the title compound as an orange oil in 75% yield, 1.1 g.

¹HNMR(400 MHz, CDCl₃) δ: 2.51(t, 2H), 3.36(s, 2H), 3.66(t, 2H), 5.56(s, 2H), 7.10(d, 2H), 7.40(d, 2H), 7.52(s, 2H); LRMS APCI m/z 344 [M+H]⁺

### Preparation 28: 1-[4-(4-Chlorophenyl)-5-(2H-1,2,3-triazol-2-ylmethyl)-4H-1,2,4-triazol-3-yl]piperidin-4-one

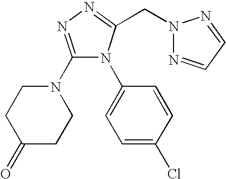

A solution of the product of preparation 26 (3.5 g, 9.74 mmol) in dimethylsulfoxide (40 ml) was cooled to 5° C. Triethylamine (10.8 ml, 77.9 mmol) and sulphur trioxide pyridine complex (4.64 g, 29.2 mmol) were added, and the mixture was stirred for 18 hours, allowing the temperature to rise to 25° C. The reaction mixture was then diluted with ethyl acetate (150 ml) and water (100 ml) and the aqueous layer was separated and re-extracted with ethyl acetate (2×75 ml). The combined organic solutions were washed with water (3×150 ml) and brine (2×150 ml), dried over magnesium sulfate and concentrated in vacuo to afford the title compound in 89% yield, 3.1 g.

¹HNMR(400 MHz, CDCl₃) δ: 2.44(m, 4H), 3.99(m, 4H), 5.60(s, 2H), 7.15(d, 2H), 7.42(d, 2H), 7.52(s, 2H); LRMS APCI m/z 358 [M+H]⁺

### Preparation 29: 1-[4-(4-Chlorophenyl)-5-(2H-1,2,3-triazol-2-ylmethyl)-4H-1,2,4-triazol-3-yl]-4-[(dimethylamino)methylene]pyrrolidin-3-one

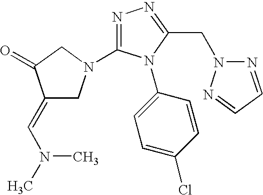

A solution of the product of preparation 27 (1.1 g, 3.2 mmol) in N,N-dimethylacetamide dimethyl acetal (3 ml, 22.4 mmol) was heated under reflux for 2 hours. The solution was then cooled to room temperature and concentrated in vacuo. Trituration of the residue with diethyl ether afforded the title compound as a red solid in 71 % yield, 905 mg.

¹HNMR(400 MHz, CDCl₃) δ: 3.10(s, 6H), 3.24(s, 2H), 4.79(s, 2H), 5.52(s, 2H), 7.05(d, 2H), 7.28(s, 1H), 7.35(d, 2H), 7.51 (s, 2H); LRMS APCI m/z 399 [M+H]⁺

### Preparation 30: 1-[4-(4-chlorophenyl)-5-(2H-1,2,3-triazol-2-ylmethyl)-4H-1,2,4-triazol-3-yl]-3-[(dimethylamino)methylene]piperidin-4-one

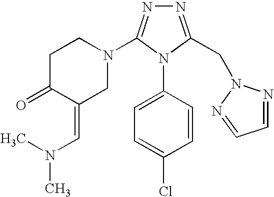

A solution of the product of preparation 28 (3.1 g, 8.67 mmol) in N,N-dimethylacetamide dimethyl acetal (8.05 ml, 60.7 mmol) was heated under reflux for 4 hours. The solution was then cooled to room temperature and concentrated in vacuo. Re-crystallisation of the residue from ethyl acetate afforded the title compound as a yellow solid in 17% yield, 600 mg.

¹HNMR(400 MHz, CDCl₃) δ: 2.19(t, 2H), 3.04(s, 6H), 3.15(t, 2H), 4.39(s, 2H), 5.59(s, 2H), 7.13(d, 2H), 7.40(d, 2H), 7.47(s, 1H), 7.51(s, 2H); LRMS APCI m/z 413 [M+H]⁺

### Preparation 31: 6-Benzyl-5H-pyrrolo[3,4-b]pyridine-5,7(6H)-dione

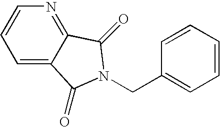

Benzylamine (3.85 ml, 35.2 mmol) was added to a suspension of 2,3-pyridinedicarboxylic anhydride (5 g, 33.5 mmol), in acetic acid (50 ml), and the mixture was heated under reflux for 18 hours. It was then cooled to room temperature, concentrated in vacuo and the residue was triturated with diethyl ether to afford the title compound as a white solid in 57% yield, 4.52 g.

¹HNMR(400 MHz, CDCl₃) δ: 4.79(s, 2H), 7.27(m, 1H), 7.32(m, 4H), 7.78(dd, 1H), 8.29(d, 1H), 8.96(d, 1H); LRMS APCI m/z 239 [M+H]⁺

### Preparation 32: 6-Benzyl-6,7-dihydro-5H-pyrrolo[3,4-b]pyridine

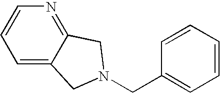

Lithium aluminium hydride (1M in diethyl ether, 37.8 ml, 37.8 mmol) was added dropwise to a suspension of the product of preparation 31 (4.5 g, 18.9 mmol), in diethyl ether (45 ml), and the mixture was stirred at room temperature for 3 hours. It was then cooled to 0° C., quenched with saturated sodium sulfate solution (50 ml) and diluted with dichloromethane (50 ml). The mixture was then filtered through Celite®, washing through with diethyl ether (50 ml) and dichloromethane (50 ml). The filtrate layers were separated and the organic solution was concentrated in vacuo. Purification of the residue by column chromatography on silica gel, eluting with dichloromethane:methanol, 95:5, afforded the title compound in 51% yield, 2.02 g.

¹HNMR(400 MHz, CD₃OD) δ: 3.94(s, 4H), 3.98(s, 2H), 7.36(m, 6H), 7.68(d, 1H), 8.32(d, 1H); LRMS APCI m/z 211 [M+H]⁺

### Preparation 33: 6,7-Dihydro-5H-pyrrolo[3,4-b]pyridine

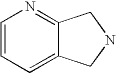

The product of preparation 32 (2 g, 9.52 mmol) and 5% Pd/C (300 mg) were stirred in ethanol (20 ml), under 50 psi of hydrogen gas, at 60° C. for 3 hours. The reaction mixture was then filtered through Arbocel® and the filtrate was concentrated in vacuo to afford the title product as a brown oil in quantitative yield. LRMS APCI m/z 121 [M+H]⁺

### Preparation 34: N-(4-Chlorophenyl)-5,7-dihydro-6H-pyrrolo[3,4-b]pyridine-6-carbothioamide

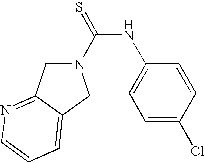

4-Chlorophenyl isothiocyanate (2.73 g, 16.2 mmol) was added dropwise to a solution of the product of preparation 33 (2.3 g, 14.7 mmol) and triethylamine (6.13 ml, 44.1 mmol), in ethanol (20 ml), and the mixture was stirred at room temperature for 18 hours. It was then concentrated in vacuo and the residue was triturated with diethyl ether. The residue was suspended in dichloromethane (100 ml), washed with sodium hydrogen carbonate solution (70 ml), dried over magnesium sulfate and concentrated in vacuo to afford the title compound in 28% yield, 1.2 g.

¹HNMR(400 MHz, CD₃OD) δ: 5.04(s, 2H), 5.11 (s, 2H), 7.31(m, 1H), 7.34(d, 2H), 7.43(d, 2H). 7.85(d, 1 H), 8.48(d, 1 H); LRMS APCI m/z 290 [M+H]⁺

### Preparation 35: Methyl N-(4-chlorophenyl)-5,7-dihydro-6H-pyrrolo[3,4-b]pyridine-6-carbimidothioate

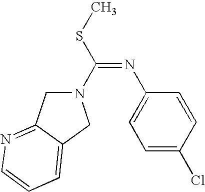

Potassium tert-butoxide (4.64 mg, 4.15 mmol) was added to an ice-cold solution of the product of preparation 34 (1.2 g, 4.15 mmol) in tetrahydrofuran (15 ml) and the mixture was stirred for 10 minutes. Methyl p-toluenesulfonate (771 mg, 4.15 mmol) was then added, and the mixture was stirred for a further 72 hours at room temperature. It was then diluted with ether (50 ml) and washed with water (2×50 ml). The organic solution was dried over magnesium sulfate and concentrated in vacuo to afford the title compound as a brown oil in quantitative yield. LRMS APCI m/z 304 [M+H]⁺

### Preparation 36: tert-Butyl diprop-2-yn-1-ylcarbamate

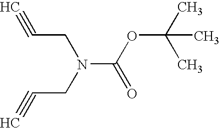

A solution of (2-propynyl)carbamic acid tert-butyl ester (16 g, 100 mmol) [J. Med. Chem. 2005, 48, 224] in N,N-dimethylformamide (200 ml) was cooled to 0° C., sodium hydride (60% dispersion in mineral oil, 4.13 g, 100 mmol) was then added and the mixture was stirred for 30 minutes. A solution of 3-bromoprop-1-yne (80% in toluene, 15.3 ml, 100 mmol), in N,N-dimethylformamide (50 ml), was added dropwise and the mixture was stirred for a further 48 hours, allowing the temperature to rise to 25° C. The reaction mixture was partitioned between ethyl acetate (200 ml) and water (300 ml), and the aqueous layer was then separated and re-extracted with ethyl acetate (200 ml). The combined organic solutions were washed with water (3×100 ml), dried over magnesium sulfate and concentrated in vacuo to afford the title compound in 73% yield, 14 g.

¹HNMR(400 MHz, CDCl₃) δ: 1.47(s, 9H), 2.22(s, 2H), 4.16(s, 4H).

### Preparation 37: tert-Butyl 5-(hydroxymethyl)-1,3-dihydro-2H-isoindole-2-carboxylate

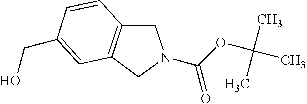

Prop-2-yn-1-ol (4.82 ml, 80 mmol) was added dropwise to an ice-cooled solution of the product of preparation 36 (4 g, 20 mmol) in ethanol (100 ml). Tris(triphenylphosphine)rhodium(l) chloride (380 mg, 0.41 mmol) was then added and the mixture was stirred at 4° C. for 2 hours. Further tris(triphenylphosphine)rhodium(I) chloride (195 mg, 0.21 mmol) was added and the reaction mixture was stirred at room temperature for a further 18 hours. Additional tris(triphenylphosphine)rhodium(l) chloride (385 mg, 0.41 mmol) was added and the mixture was heated at 40° C. for 18 hours. The reaction mixture was then concentrated in vacuo and the residue was purified by column chromatography on silica gel, eluting with pentane:ethyl acetate 100:0 to 50:50, to afford the title compound as a brown solid in 64% yield, 3.2 g.

¹HNMR(400 MHz, DMSO-d₆) δ: 1.43(s, 9H), 4.46(d, 2H), 4.54(m, 4H), 5.18(t, 1H), 7.20(d, 1H), 7.22(s, 1H), 7.25(d, 1H); GCMS m/z 250 [M+H]⁺

### Preparation 38: 2,3-Dihydro-1H-isoindol-5-ylmethanol hydrochloride

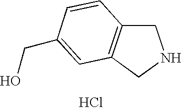

A solution of the product of preparation 37 (1.5 g, 6.02 mmol) in hydrochloric acid (4M in dioxan, 10 ml) was stirred for 18 hours, at room temperature. The reaction mixture was then concentrated in vacuo to afford the title compound as a brown solid in quantitative yield.

¹HNMR(400 MHz, CD₃OD) δ: 4.61(m, 4H), 4.64(s, 2H), 7.37-7.47(m, 3H); LRMS APCI m/z 150 [M+H]⁺

### Preparation 39: N-(4-Chlorophenyl)-5-(hydroxymethyl)-1,3-dihydro-2H-isoindole-2-carbothioamide

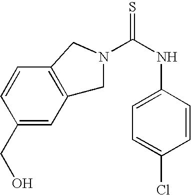

A mixture of the product of preparation 38 (1.2 g, 6.47 mmol) and triethylamine (2.69 ml, 19.4 mmol), in ethanol (10 ml), was stirred for 5 minutes at room temperature. 4-Chlorophenyl isothiocyanate (1.09 g, 6.47 mmol) was then added and the mixture was stirred at room temperature for 3 hours. The solvent was evaporated under reduced pressure and the residue was triturated with diethyl ether to afford the title compound as a brown solid in 90% yield, 1.85 g. LRMS APCI m/z 319 [M+H]⁺

### Preparation 40: Methyl N-(4-chlorophenyl)-5-(hydroxymethyl)-1,3-dihydro-2H-isoindole-2-carbimidothioate

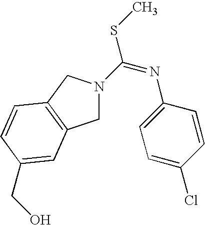

The title compound was prepared from the product of preparation 39 and methyl p-toluenesulfonate, using the same method as that described for preparation 15, in quantitative yield.

¹HNMR(400 MHz, CDCl₃) δ: 2.01(s, 3H), 4.70(s, 2H), 4.90(s, 4H), 6.98(d, 2H), 7.20-7.28(m, 3H), 7.35(d, 2H); LRMS APCI m/z 333 [M+H]⁺

### Preparation 41: 2-[4-(4-chlorophenyl)-5-(2H-1,2,3-triazol-2-ylmethyl)-4H-1,2,4-triazol-3-yl]isoindoline-5-carbaldehyde

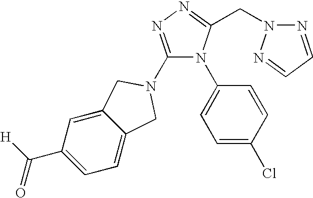

1,1,1-Triacetoxy-1,1-dihydro-1,2-benziodoxol-3(1H)-one (328 mg, 0.77 mmol) was added to an ice-cold solution of the product of example 10 (210 mg, 0.52 mmol) in dichloromethane (10 ml) and the mixture was stirred for 2 hours at room temperature. 10% Sodium thiosulfate (10 ml) was added dropwise, followed by saturated sodium hydrogen carbonate solution (10 ml) and diethyl ether (10 ml), and the mixture was stirred for a further 15 minutes, at room temperature. It was then diluted with dichloromethane (40 ml) and the organic layer was separated, dried over magnesium sulfate and concentrated in vacuo to afford the title compound as a yellow solid in 96% yield, 200 mg.

¹HNMR(400 MHz, CDCl₃) δ: 4.57(s, 2H), 4.68(s, 2H), 5.56(s, 2H), 7.23(d, 2H), 7.36(d, 1H), 7.45(d, 2H), 7.53(s, 2H), 7.69(s, 1H), 7.77(d, 1 H), 9.96(s, 1H); LRMS APCI m/z 406 [M+H]⁺

### Preparation 42: 2-[4-(4-Chlorophenyl)-5-methyl-4H-1,2,4-triazol-3-yl]isoindoline-5-carbaldehyde

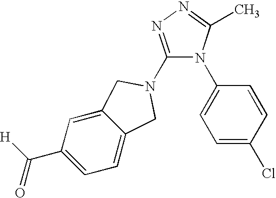

The title compound was prepared from the product of example 12 and 1,1,1-triacetoxy-1,1-dihydro-1,2-benziodoxol-3(1H)-one, using the same preparation as that described for preparation 41, as a yellow solid in 98% yield.

¹HNMR(400 MHz, CDCl₃) δ: 2.14(s, 3H), 4.51(s, 2H), 4.61(s, 2H), 7.30(d, 1H), 7.37(d, 2H), 7.53(d, 2H), 7.64(s, 1H), 7.72(d, 1H), 9.90(s, 1H); LRMS APCI m/z 339 [M+H]⁺

### Preparation 43: 2-[4-(4-Chlorophenyl)-5-methyl-4H-1,2,4-triazol-3-yl]isoindoline-5-carboxylic acid

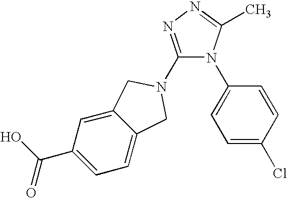

The title compound was prepared from the product of preparation 42, using the same method as that described for example 14, as a yellow oil in 15% yield.

¹HNMR(400 MHz, CD₃OD) δ: 2.17(s, 3H), 4.55(m, 4H), 7.33(d, 1H), 7.60(d, 2H), 7.69(d, 2H), 7.88(s, 1H), 7.94(d, 1H); LRMS APCI m/z 355 [M+H]⁺

### Preparation 44: 1,1,1,2-Tetramethoxy-ethane

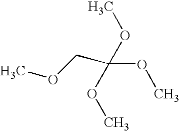

Methoxyacetonitrile (50.0 g, 704 mmol) was dissolved in a mixture of methanol (34 ml) and diethyl ether (210 ml), and the mixture was cooled to 0° C. Hydrogen chloride gas was bubbled through the solution for 20 minutes, and then the reaction mixture was stirred at room temperature for 2 hours. Hydrogen chloride gas was bubbled through the mixture for a second time and it was then allowed to stand at −15° C. for 18 hours. The mixture was then filtered and the resulting white residue was washed with diethyl ether (150 ml), dissolved in methanol (340 ml) and stirred for 90 minutes. The solution was then diluted with further diethyl ether (370 ml), heated under reflux for 6 hours and then left to stand at room temperature for 18 hours. Additional diethyl ether (200 ml) was added and the mixture was filtered off. The filtrate was washed with 10% sodium carbonate solution, dried over magnesium sulfate and concentrated in vacuo to yield the title product in 32% yield, 34.5 g. ¹HNMR(CDCl₃, 400 MHz) δ: 3.29(s, 9H), 3.39(s, 3H), 3.50(s, 2H)

### Preparation 45: 2-[5-(Methoxymethyl)-1,3,4-oxadiazol-2-yl]imidazo[1,2-a]pyridine

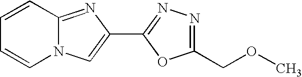

1,1,1,2-Tetramethoxy-ethane (preparation 44), (410 mg, 2.72 mmol) was added to a solution of imidazo[1,2-a]pyridine-2-carboxylic acid hydrazide (400 mg, 2.27 mmol) [Tetrahedron, 2002, 58, 295], in N,N-dimethylformamide (15 ml), and the reaction mixture was heated at 60° C. for 18 hours. Further 1,1,1,2-tetramethoxy-ethane (preparation 44), (342 mg, 2.27 mmol) was added and mixture was heated at 80° C. for a further 3 hours. It was then concentrated in vacuo and the residue was taken up in toluene (15 ml). para-Toluenesulfonic acid (43 mg) was added and the reaction mixture was heated at 110° C. for 3 days. It was then concentrated in vacuo and the residue was purified by column chromatography on silica gel, eluting with dichloromethane:methanol:0.88 ammonia, 90:10:1 to 40:60:6, to afford the title compound in 38% yield, 200 mg.

¹HNMR(400 MHz, CDCl₃) δ: 3.49(s, 3H), 4.75(s, 2H), 6.93(m, 1H), 7.32(m, 1H), 7.73(d, 1H), 8.20(d, 1H), 8.31 (s, 1H); LRMS ESI m/z 231 [M+H]⁺

### EXAMPLE 1

### 2-[4-(4-Chlorophenyl)-5-(methoxymethyl)-4H-1,2,4-triazol-3-yl]isoindoline

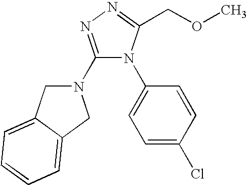

A mixture of the product of preparation 9 (500 mg, 1.65 mmol), methoxyacetohydrazide (190 mg, 1.82 mmol) and trifluoroacetic acid (61 μl, 0.83 mmol), in tetrahydrofuran (20 ml), was heated under reflux for 6 hours. The cooled mixture was then diluted with ethyl acetate (100 ml) and washed with sodium hydrogen carbonate solution (100 ml). The organic solution was dried over magnesium sulfate, concentrated in vacuo and the residue was triturated with diethyl ether to afford the title compound as a pale brown solid in 60% yield, 340 mg.

¹HNMR(400 MHz, CDCl₃) δ: 3.30(s, 3H), 4.30(s, 2H), 4.54(s, 4H), 6.98(m, 2H), 7.13(m, 2H), 7.45(m, 2H), 7.55(m, 2H); LRMS APCI m/z 341 [M+H]⁺

### EXAMPLE 2

### 2-[4-(4-Chlorophenyl)-5-methyl-4H-1,2,4-triazol-3-yl]isoindoline

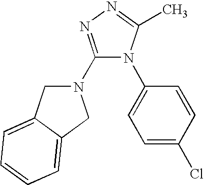

The title compound was prepared from the product of preparation 9 and acethydrazide, using the same method as that described for example 1, as a pale brown solid in 38% yield.

¹HNMR(400 MHz, CDCl₃) δ: 2.19(s, 3H), 4.48(s, 4H), 6.98(m, 2H), 7.18(m, 2H), 7.23(m, 2H), 7.32(m, 2H), 7.55(m, 2H); LRMS APCI m/z 311 [M+H]⁺

### EXAMPLE 3

### 2-[4-(4-Chlorophenyl)-5-methyl-4H-1,2,4-triazol-3-yl]-2,3,4,9-tetrahydro-1H-L-carboline

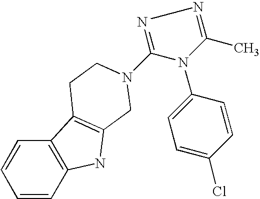

The title compound was prepared from the product of preparation 10 and acethydrazide, using the same method as that described for example 1, as white solid in 62% yield.

¹HNMR(400 MHz, CDCl₃) δ: 2.31(s, 3H), 2.55(m, 2H), 3.19(m, 2H), 4.65(m, 2H), 7.05(m, 1H), 7.14(m, 1H), 7.30(d, 2H), 7.39(d, 1H), 7.50(d, 1H), 7.55(m, 1H), 9.25-9.42(bs, 1H); LRMS APCI m/z 364 [M+H]⁺

### EXAMPLE 4

### 1′-[4-(4-Chlorophenyl)-5-methyl-4H-1,2,4-triazol-3-yl]-3H-spiro[2-benzofuran-1,4′-piperidin]-3-one

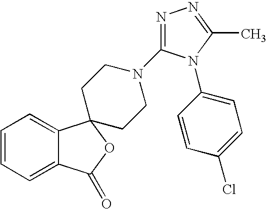

The title compound was prepared from the product of preparation 11 and acethydrazide, using the same method as that described for example 1, as a solid in 60% yield.

¹HNMR(400 MHz, CDCl₃) δ: 1.58(m, 2H), 2.17-2.29(m, 5H), 3.37(m, 4H), 7.27(m, 2H), 7.39(m, 1H), 7.51(m, 3H), 7.65(m, 1H), 7.85(m, 1H); LRMS APCI m/z 395 [M+H]⁺

### EXAMPLE 5

### 1′-[4-(4-chlorophenyl)-5-methyl-4H-1,2,4-triazol-3-yl]spiro[isoindole-1,4′-piperidin]-3(2H)-one

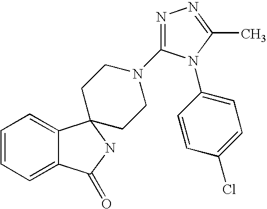

A mixture of the product of preparation 13 (2.3 g, 5.97 mmol), acethydrazide (663 mg, 8.96 mmol) and trifluoroacetic acid (23 μl, 8.96 mmol), in tetrahydrofuran (50 ml), was heated under reflux for 45 minutes. The cooled mixture was then diluted with sodium hydroxide solution (50 ml) and extracted with ethyl acetate (2×70 ml). The combined organic solutions were washed with brine (100 ml), dried over magnesium sulfate and concentrated in vacuo. Purification of the residue by column chromatography on silica gel, eluting with dichloromethane:methanol:0.88 ammonia, 93:7:1, afforded the title compound as a white foam in 78% yield, 1.84 g.

¹HNMR(400 MHz, CDCl₃) δ: 1.75(m, 2H), 1.90(m, 2H), 2.35(s, 3H), 3.30(m, 2H), 3.55(m, 2H), 6.90(d, 1H), 7.00(m, 1H), 7.20(m, 1H), 7.35(m, 2H), 7.45(d, 1H), 7.55(m, 2H), 8.55(s,1 H); LRMS APCI m/z 394 [M+H]⁺.

### EXAMPLE 6

### 1′-[4-(4-Chlorophenyl)-5-methyl-4H-1,2,4-triazol-3-yl]-3H-spiro[2-benzofuran-1,4′-piperidine]

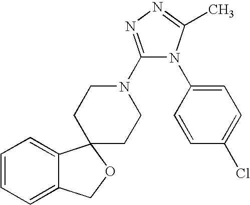

A suspension of the product of example 4 (0.2 g, 0.5 mmol) in tetrahydrofuran (1.5 ml) was cooled to 5° C. and treated with 1M borane-tetrahydrofuran complex (1M, 1.5 ml, 1.5 mmol). The mixture was stirred at 5° C. for 15 minutes, at room temperature for 30 minutes and then heated under reflux for 18 hours. It was then cooled to room temperature, diluted with 6M hydrochloric acid (3 ml) and re-heated to reflux for a further 3 hours. The reaction mixture was then cooled to room temperature, basified with 1 M sodium hydroxide (30 ml) and extracted with dichloromethane (2×30 ml). The combined organic solutions were dried over sodium sulfate, concentrated in vacuo and the residue was purified by column chromatography on silica gel, eluting with dichloromethane:methanol:0.88 ammonia, 90:10:1, to afford the title compound in 26% yield, 50 mg.

¹HNMR(400 MHz, CDCl₃) δ: 1.64(m, 2H), 1.83-1.94(m, 2H), 2.23(s, 3H), 3.27(m, 4H), 5.01(s, 2H), 7.09(m, 1H), 7.18(m, 1H), 7.21-7.52(m, 4H), 7.62(d, 2H); LRMS APCI m/z 381 [M+H]⁺

### EXAMPLE 7

### 1′-[4-(4-Chlorophenyl)-5-methyl-4H-1,2,4-triazol-3-yl]-2-methylspiro[isoindole-1,4′-piperidin]-3(2H)-one

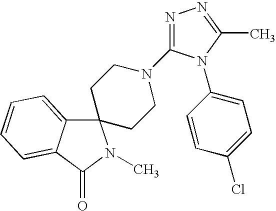

A solution of the product of example 5 (250 mg, 0.64 mmol) in tetrahydrofuran (2 ml) was added to a suspension of sodium hydride (60% dispersion in mineral oil, 51 mg, 1.28 mmol) in tetrahydrofuran (5 ml) and the mixture was stirred at room temperature for 30 minutes. Methyl iodide (50 μl, 0.8 mmol) was then added and the reaction mixture was stirred at room temperature for a further 2 hours. It was then poured onto ice and was extracted with ethyl acetate (2×50 ml). The combined organic solution was washed with brine (25 ml), dried over magnesium sulfate, concentrated in vacuo and the residue was purified by column chromatography on silica gel, eluting with 95:5:0.5, to afford the title compound as a white foam in 71% yield, 184 mg.

¹HNMR(400 MHz, CDCl₃) δ: 1.70(m, 2H), 1.85(m, 2H), 2.25(s, 3H), 3.15(s, 3H), 3.30(m, 2H), 3.50(m, 2H), 6.90(d, 1H), 7.05(m, 1H), 7.30(m, 3H), 7.40(d, 1H), 7.55(d, 2H); LRMS APCI m/z 408 [M+H]⁺

### EXAMPLE 8

### 2-[4-(4-Chlorophenyl)-5-(2H-1,2,3-triazol-2-ylmethyl)-4H-1,2,4-triazol-3-yl]isoindoline

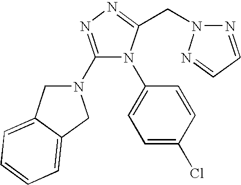

The title compound was prepared form the products of preparations 9 and 18, using the same method as that described for example 1. The crude compound was purified by column chromatography on silica gel, eluting with ethyl acetate:methanol:0.88 ammonia, 90:10:1, to afford the desired product in 16% yield.

¹HNMR(400 MHz, CDCl₃) δ: 4.49(s, 4H), 5.57(s, 2H), 7.15(m, 4H), 7.24(m, 2H), 7.41(d, 2H), 7.52(s, 2H); LRMS APCI m/z 378 [M+H]⁺

### EXAMPLE 9

### 6-[4-(4-Chlorophenyl)-5-(2H-1,2,3-triazol-2-ylmethyl)-4H-1,2,4-triazol-3-yl]-6,7-dihydro-5H-pyrrolo[3,4-b]pyridine

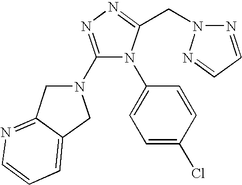

A mixture of the product of preparation 35 (750 mg, 2.48 mmol), preparation 18 (419 mg, 2.97 mmol) and trifluoroacetic acid (94 μl, 1.24 mmol), in tetrahydrofuran (10 ml), was heated under reflux for 18 hours. Further trifluoroacetic acid (282 μl, 3.72 mmol) was added and heating was continued for a further 18 hours. The cooled reaction mixture was then partitioned between dichloromethane (100 ml) and water (70 ml) and passed through an Isolute® hydrophobic membrane. The organic solution was concentrated in vacuo and the residue was purified by column chromatography on silica gel, eluting with dichloromethane:methanol, 100:0 to 90:10, to afford the title compound as a brown glass in 3% yield, 35 mg.

¹HNMR(400 MHz, CDCl₃) δ: 4.28(s, 2H), 4.78(s, 2H), 5.56(s, 2H), 7.13(d, 2H), 7.18(m, 1H), 7.40(d, 2H), 7.53(s, 2H), 7.56(d, 1H), 8.41(d, 1H); LRMS APCI m/z 379 [M+H]⁺

### EXAMPLE 10

### {2-[4-(4-Chlorophenyl)-5-(2H-1,2,3-triazol-2-ylmethyl)-4H-1,2,4-triazol-3-yl]-2,3-dihydro-1H-isoindol-5-yl}methanol

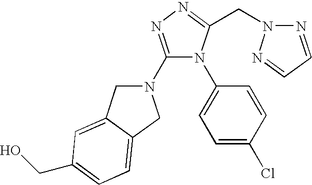

A mixture of the product of preparation 40 (1 g, 3.02 mmol), preparation 18 (509 mg, 3.61 mmol) and trifluoroacetic acid (115 μl, 1.52 mmol), in tetrahydrofuran (10 ml), was heated under reflux for 18 hours. The cooled mixture was then partitioned between dichloromethane (100 ml) and water (80 ml) and passed through an Isolute® hydrophobic membrane. The organic solution was concentrated in vacuo and the residue was purified by column chromatography on silica gel, eluting with dichloromethane:methanol, 100:0 to 90:10, to afford the title compound in 34% yield, 210 mg

¹HNMR(400 MHz, CDCl₃) δ: 4.50(m, 4H),4.65(s, 2H), 5.55(s, 2H), 7.12-7.22(m, 5H), 7.42(d, 2H), 7.52(s, 2H); LRMS APCI m/z 408 [M+H]⁺

### EXAMPLE 11

### 1′-[4-(4-chlorophenyl)-5-methyl-4H-1,2,4-triazol-3-yl]-2,3-dihydrospiro[indene-1,4′-piperidine]

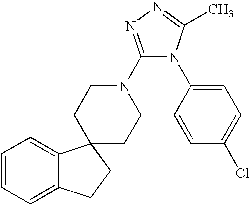

A mixture of the product of preparation 12 (900 mg, 2.42 mmol) and acethydrazide (448 mg, 6.0 mmol) in acetic acid (20 ml) was heated under reflux for 18 hours. The cooled mixture was then partitioned between ethyl acetate (100 ml) and 2M with sodium hydrogen carbonate solution (150 ml). The aqueous layer was separated, extracted with ethyl acetate (2×50 ml) and the combined organic solutions were washed with brine (100 ml), dried over magnesium sulfate and concentrated in vacuo. The residue was purified by column chromatography on silica gel, eluting with ethyl acetate followed by ethyl acetate:methanol, 95:5, followed by dichloromethane:methanol:0.88 ammonia, 90:10:1. The appropriate fractions were evaporated under reduced pressure and the residue was triturated with diethyl ether and re-crystallised from ethyl acetate to afford the title compound as a solid in 20% yield, 187 mg.

¹HNMR(400 MHz, CDCl₃) δ: 1.45(d, 2H), 1.78(m, 2H), 2.02(m, 2H), 2.25(s, 3H), 2.88(t, 2H), 3.00(t, 2H), 3.26(d, 2H), 7.15(m, 4H), 7.31(d, 2H), 7.54(d, 2H); LRMS ESI m/z 379 [M+H]⁺

### EXAMPLES 12 AND 13

A mixture of the product of preparation 40 (1 g, 3.02 mmol), acethydrazide (446 mg, 6.04 mmol) and trifluoroacetic acid (115 μl, 1.52 mmol), in tetrahydrofuran (10 ml), was heated under reflux for 18 hours. The cooled mixture was then partitioned between dichloromethane (100 ml) and water (80 ml) and passed through an Isolute® hydrophobic membrane. The organic solution was concentrated in vacuo and the residue was purified by column chromatography on silica gel, eluting with dichloromethane:methanol, 100:0 to 90:10, to afford the product of example 12. Further elution using the same gradient then afforded the product of example 13.

### EXAMPLE 12

### {2-[4-(4-Chlorophenyl)-5-methyl-4H-1,2,4-triazol-3-yl]-2,3-dihydro-1H-isoindol-5-yl}methanol

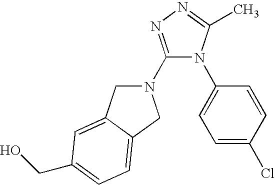

¹HNMR(400 MHz, CDCl₃) δ: 2.18(s, 3H), 4.48(m, 4H), 4.66(s, 2H), 7.19(m, 3H), 7.35(d, 2H), 7.55(d, 2H); LRMS APCI m/z 341 [M+H]⁺

### EXAMPLE 13

### 2-[4-(4-Chlorophenyl)-5-methyl-4H-1,2,4-triazol-3-yl]-5-(methoxymethyl)isoindoline

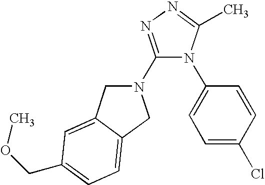

¹HNMR(400 MHz, CDCl₃) δ: 2.17(s, 3H), 3.36(s, 3H), 4.41(s, 2H), 4.46(m, 4H), 7.15(m, 3H), 7.31(d, 2H), 7.53(d, 2H); LRMS APCI m/z 354 [M+H]⁺

### EXAMPLE 14

### 2-[4-(4-Chlorophenyl)-5-(2H-1,2,3-triazol-2-ylmethyl)-4H-1,2,4-triazol-3-yl]isoindoline-5-carboxylic acid

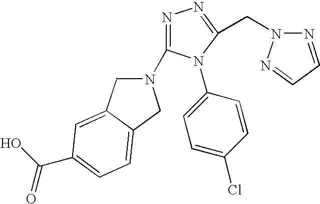

2-Methyl-2-butene (12.4 ml, 24.7 mmol) was added to a solution of the product of preparation 41 (200 mg, 0.49 mmol) in ^{t}butanol (25 ml), and the reaction mixture was stirred for 5 minutes. A solution of sodium chlorite (267 mg, 2.96 mmol) and sodium phosphate (533 mg, 4.44 mmol) in water (5 ml) was then added dropwise and the reaction mixture was stirred at room temperature for a further 2 hours. Sodium sulfite solution was then added, until starch/potassium iodide analysis showed that all of the sodium chlorite and sodium phosphate had been removed. Then the mixture was diluted with dichloromethane (50 ml) and water (50 ml). The organic layer was separated, dried over sodium sulfate and concentrated in vacuo to afford the title compound as a yellow oil in 13% yield, 172 mg.

¹HNMR(400 MHz, CD₃OD) δ: 4.54(m, 4H), 5.61(s, 2H), 7.32(m, 3H), 7.52(d, 2H), 7.59(s, 2H), 7.87(s,1 H), 7.92(d, 1H); LRMS APCI m/z 422 [M+H]⁺

### EXAMPLE 15

### 3-[4-(4-Chlorophenyl)-5-methyl-4H-1,2,4-triazol-3-yl]-2,3,4,5-tetrahydro-1H-3-benzazepine

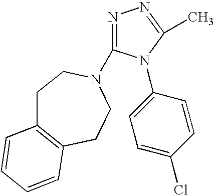

The title compound was prepared from the product of preparation 14 and acethydrazide, using the same method as that described for example 8, as a pale yellow solid in 49% yield.

¹HNMR(400 MHz, CDCl₃) δ: 2.25(s, 3H), 2.90(m, 4H), 3.05(m, 4H), 7.05(m, 2H), 7.13(m, 2H), 7.25(d, 2H), 7.55(d, 2H); LRMS APCI m/z 339 [M+H]⁺

### EXAMPLES 16-18

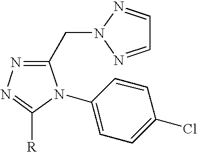

O-(1H-Benzotriazol-1-yl)-N,N,N′,N′-tetramethyluronium hexafluorophosphate (112 mg, 0.30 mmol), triethylamine (207 μl, 1.49 mmol) and the product of preparation 24 (80 mg, 0.25 mmol) were added to a solution of the appropriate carboxylic acid (RCOOH, 0.28 mmol) in acetonitrile (1 ml), and the reaction mixture was stirred at room temperature for 18 hours. It was then heated under reflux for a further 18 hours before being concentrated in vacuo. The residues were diluted with dichloromethane (20 ml), washed with saturated sodium carbonate (20 ml), passed through a hydrophobic membrane and concentrated in vacuo. Purification of the residue by HPLC using a Phenomenex Luna C18 system, eluting with water/acetonitrile/trifluoroacetic acid (5:95:0.5):acetonitrile, 95:5 to 5:95, afforded the title compounds.

| Ex | | | |
|---|---|---|---|
| No | R | Data | Yield |
| 16 | 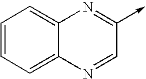 | ¹HNMR (400 MHz, CDCl₃) δ: 5.79 (s, 2 H),7.13 (d, 2 H), 7.42 (d, 2 H), 7.46 (d, 1 H), 7.56 (s,2 H), 7.67 (m, 1 H), 7.76 (m, 1 H), 8.10 (d, 1 H),9.78 (s, 1 H); LRMS APCI m/z 389 [M + H]⁺ | 21% |
| 17 | 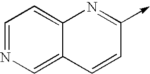 | ¹HNMR (400 MHz, CDCl₃) δ: 5.82 (s, 2 H),7.15 (d, 2 H), 7.46 (d, 2 H), 7.56 (d, 1 H), 7.58 (s,2 H), 8.68 (d, 2 H), 8.81 (d, 1 H), 9.68 (s, 1 H);LRMS APCI m/z 389 [M + H]⁺ | 25% |
| 18 | 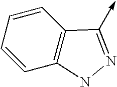 | ¹HNMR (400 MHz, CDCl₃) δ: 5.30 (s, 2 H),7.11 (d, 2 H), 7.32 (d, 1 H), 7.38 (d, 2 H), 7.46 (m,2 H), 7.55 (s, 2 H), 8.48 (d, 1 H); LRMS: APCIm/z 377 [M + H]⁺ | 6% |

### EXAMPLE 19

### 5-[4-(4-Chlorophenyl)-5-(2H-1,2,3-triazol-2-ylmethyl)-4H-1,2,4-triazol-3-yl]-1-methyl-1,4,5,6-tetrahydropyrrolo[3,4-c]pyrazole

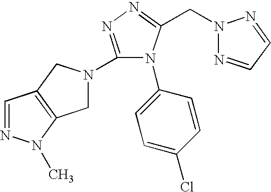

A mixture of the product of preparation 29 (150 mg, 0.38 mmol) and methylhydrazine (30 μl, 0.57 mmol) in methanol (3 ml) was heated under reflux for 3 hours. The reaction mixture was then concentrated in vacuo and the residue was partitioned between ethyl acetate (20 ml) and water (20 ml). The organic layer was separated, dried over magnesium sulfate and concentrated in vacuo. The residue was triturated with diethyl ether and the residue was purified by column chromatography on silica gel, eluting with dichloromethane:methanol, 100:0 to 90:10, to afford the title compound in 17% yield, (26 mg).

¹HNMR(400 MHz, CDCl₃) δ: 3.79(s, 3H), 4.13(s, 2H), 4.82(s, 2H), 5.54(s, 2H), 7.12(s, 1H), 7.31(d, 2H), 7.45(d, 2H), 7.53(s, 2H).

### EXAMPLE 20

### 6-[4-(4-Chlorophenyl)-5-(2H-1,2,3-triazol-2-ylmethyl)-4H-1,2,4-triazol-3-yl]-6,7-dihydro-5H-pyrrolo[3,4-d]pyrimidine

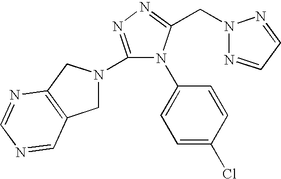

A mixture of the product of preparation 29 (120 mg, 0.30 mmol), formamidine hydrochloride (121 mg, 1.50 mmol) and triethylamine (251 μl, 1.8 mmol), in ethanol (5 ml), was heated under reflux for 18 hours. The reaction mixture was then cooled to room temperature, concentrated in vacuo and the residue was diluted with dichloromethane (40 ml) and washed with sodium hydrogen carbonate solution (40 ml). The organic solution was dried over magnesium sulfate, concentrated in vacuo and the residue was purified by column chromatography on silica gel, eluting with dichloromethane:methanol, 100:0 to 90:10, to afford the title compound as a pale orange solid in 36% yield, 41.3 mg.

¹HNMR(400 MHz, CDCl₃) δ: 4.32(m, 2H), 4.73(m, 2H), 5.51(s, 2H), 7.12(d, 2H), 7.37(d, 2H), 7.47(s, 2H), 8.58(s,1H), 9.01 (s,1 H); LRMS APCI m/z 380 [M+H]⁺

### EXAMPLE 21

### 7-[4-(4-Chlorophenyl)-5-(2H-1,2,3-triazol-2-ylmethyl)-4H-1,2,4-triazol-3-yl]-5,6,7,8-tetrahydropyrido[3,4-d]pyrimidine

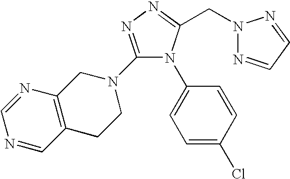

The title compound was prepared from the product of preparation 30 and formamidine hydrochloride, using the same method as that described for example 29, as a yellow solid in 50% yield.

¹HNMR(400 MHz, CDCl₃) δ: 2.76(t, 2H), 3.30(t, 2H), 4.47(s, 2H), 5.61(s, 2H), 7.16(d, 2H), 7.44 (d, 2H), 7.53(s, 2H), 8.42(s, 1H), 8.99(s, 1H); LRMS APCI m/z 394 [M+H]⁺

### EXAMPLE 22

### 6-[4-(4-Chlorophenyl)-5-(2H-1,2,3-triazol-2-ylmethyl)-4H-1,2,4-triazol-3-yl]-2-methyl-6,7-dihydro-5H-pyrrolo[3,4-d]pyrimidine

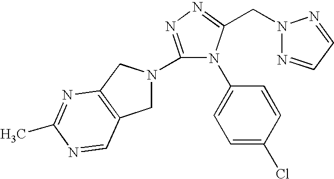

The title compound was prepared from the product of preparation 29 and acetamidine hydrochloride, using the same method as that described for example 20, as pale orange solid in 44% yield.

¹HNMR(400 MHz, CDCl₃) δ: 2.70(s, 3H), 4.31(m, 2H), 4.83(m, 2H), 5.56(s, 2H), 7.21(d, 2H), 7.42(d, 2H), 7.53(s, 2H), 8.56(s, 1H); LRMS APCI m/z 394 [M+H]⁺

### EXAMPLE 23

### 7-[4-(4-Chlorophenyl)-5-(2H-1,2,3-triazol-2-ylmethyl)-4H-1,2,4-triazol-3-yl]-2-methyl-5,6,7,8-tetrahydropyrido[3,4-d]pyrimidine

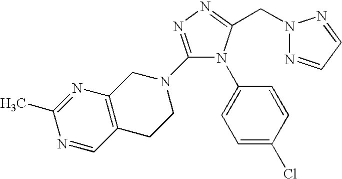

The title compound was prepared from the product of preparation 30 and acetamidine hydrochloride, using the same method as that described for example 20, as a yellow solid in 40% yield.

¹HNMR(400 MHz, CDCl₃) δ: 2.71(s, 3H), 2.78(t, 2H), 3.30(t, 2H), 4.44(s, 2H), 5.61(s, 2H), 7.16(d, 2H), 7.44(d, 2H), 7.53(s, 2H), 8.37(s, 1H); LRMS APCI m/z 408 [M+H]⁺

### EXAMPLE 24

### 5-[4-(4-Chlorophenyl)-5-(2H-1,2,3-triazol-2-ylmethyl)-4H-1,2,4-triazol-3-yl]-5,6-dihydro-4H-pyrrolo[3,4-c]isoxazole

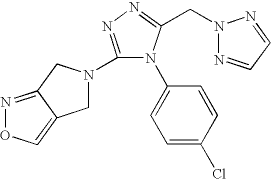

A mixture of the product of preparation 29 (120 mg, 0.30 mmol), hydroxylamine hydrochloride (32 mg, 0.45 mmol) and triethylamine (105 μl, 0.75 mmol), in methanol (5 ml), was heated under reflux for 18 hours. The reaction mixture was then cooled to room temperature, concentrated in vacuo and the residue was diluted with dichloromethane (30 ml) and washed with sodium hydrogen carbonate solution (30 ml). The organic solution was dried over magnesium sulfate, concentrated in vacuo and the residue was purified by column chromatography on silica gel, eluting with dichloromethane:methanol, 100:0 to 90:10, to afford the title compound in 14% yield, 15 mg.

¹HNMR(400 MHz, CDCl₃) δ: 3.02-3.67(m, 4H), 5.51(s, 2H), 7.04(d, 2H), 7.33(m, 3H), 7.47(s, 2H); LRMS APCI m/z 369 [M+H]⁺

### EXAMPLE 25

### 5-[4-(4-Chlorophenyl)-5-(2H-1,2,3-triazol-2-ylmethyl)-4H-1,2,4-triazol-3-yl]-2-methyl-4,5,6,7-tetrahydro-2H-pyrazolo[4,3-c]pyridine

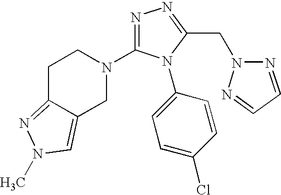

A mixture of the product of preparation 30 (150 mg, 0.36 mmol), methyl hydrazine (38 μl, 0.73 mmol) and triethylamine (152 μl, 1.09 mmol), in ethanol (4 ml), was heated under reflux for 18 hours. The reaction mixture was then cooled to room temperature, concentrated in vacuo and the residue was diluted with dichloromethane (30 ml) and washed with sodium hydrogen carbonate solution (30 ml). The reaction mixture was then passed though an Isolute® hydrophobic membrane and concentrated in vacuo to afford the title compound as a pale yellow solid in 63% yield, 90 mg.

¹HNMR(400 MHz, CDCl₃) δ: 2.51(t, 2H), 3.24(t, 2H), 3.82(s, 3H), 4.32(s, 2H), 5.59(s, 2H), 7.10(s, 1H), 7.15(d, 2H), 7.41(d, 2H), 7.51(s, 2H); LRMS APCI m/z 396 [M+H]⁺

### EXAMPLE 26

### 5-[4-(4-Chlorophenyl)-5-(2H-1,2,3-triazol-2-ylmethyl)-4H-1,2,4-triazol-3-yl]-2-isopropyl-4,5,6,7-tetrahydro-2H-pyrazolo[4,3-c]pyridine

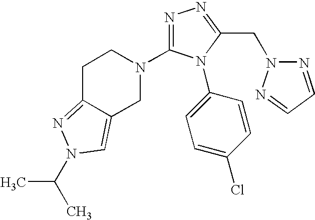

The title compound was prepared from the product of preparation 30 and isopropyl hydrazine hydrochloride (WO2004056751, p20), using the same method as that described for example 25, as a pale yellow solid in 62% yield.

¹HNMR(400 MHz, CDCl₃) δ: 1.44(d, 6H), 2.71(t, 2H), 3.53(t, 2H), 4.07(s, 2H), 4.30(m, 1H), 5.57(s, 2H), 7.16(d, 2H), 7.20(s, 1H), 7.43(d, 2H), 7.52(s, 2H); LRMS APCI m/z 424 [M+H]⁺

### EXAMPLES 27-30

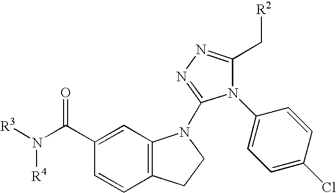

O-(1H-Benzotriazol-1-yl)-N,N,N′,N′-tetramethyluronium hexafluorophosphate (1.5 eq.), triethylamine (1.5 eq.) and the appropriate amine (1.5 eq.) were added to either the product of preparation 43 (1 eq.), or example 14 (1 eq.), in N,N-dimethylformamide (2 ml) and the mixture was stirred at room temperature for 18 hours. Further O-(1H-benzotriazol-1-yl)-N,N,N′,N′-tetramethyluronium hexafluorophosphate (2 eq.) and amine (2 eq.) were then added and the mixture was stirred for a further 72 to 120 hours. The reaction mixture was then partitioned between ethyl acetate (40 ml) and sodium hydrogen carbonate solution (30 ml) and the organic layers were separated, washed with water (3×30 ml), dried over magnesium sulfate and concentrated in vacuo. Purification of the residue by column chromatography on silica gel, eluting dichloromethane:methanol, 100:0 to 90:10, then afforded the appropriate title compound.

| Ex No | R² | Data | Yield |
|---|---|---|---|
| 27 | H | R³ = R⁴ = H | 25% |
| | | ¹HNMR (400 MHz, CD₃OD) δ: 2.16 (s, 3 H), 4.52 (s, | |
| | | 4 H), 7.32 (d, 1 H), 7.56 (d, 2 H), 7.67 (d, 2 H), 7.71 (s, | |
| | | 1 H), 7.77 (d, 1 H); LRMS APCI m/z 365 [M + H]⁺ | |
| 28 | H | R³ = R⁴ = CH₃ | 34% |
| | | ¹HNMR (400 MHz, CDCl₃) δ: 2.17 (s, 3 H), 2.94 (s, | |
| | | 3 H), 3.09 (s, 3 H), 4.64 (s, 2 H), 4.68 (s, 2 H), 7.23 (d, | |
| | | 1 H), 7.31-7.35 (m, 2 H), 7.52 (d, 2 H), 7.61 (d, 2 H); | |
| | | LRMS APCI m/z 382[M + H]⁺ | |
| 29 | 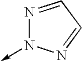 | R³ = R⁴ = H¹HNMR (400 MHz, CD₃OD) δ: 4.52 (s, 4 H), 5.60 (s,2 H), 7.32 (m, 3 H), 7.50 (d, 2 H), 7.59 (s, 2 H), 7.70 (s,1 H), 7.76 (d, 1 H); LRMS APCI m/z 421[M + H]⁺ | 27% |
| 30 | 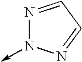 | R³ = H; R⁴ = CH₃¹HNMR (400 MHz, CD₃OD) δ: 2.88 (s, 3 H), 4.51 (s,4 H), 5.60 (s, 2 H), 7.32 (m, 3 H), 7.50 (d, 2 H), 7.58 (s,2 H), 7.63 (s, 1 H), 7.69 (d, 1 H); LRMS APCI m/z435[M + H]⁺ | 65% |

### EXAMPLE 31

### 2-[4-(4-Chlorophenyl)-5-(methoxymethyl)-4H-1,2,4-triazol-3-yl]imidazo[1,2-a]pyridine

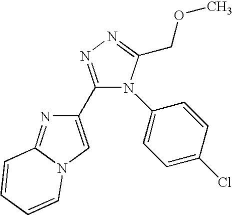

A mixture of the product of preparation 45 (190 mg, 0.83 mmol), 4-chloroaniline (126 mg, 0.99 mmol) and trifluoroacetic acid (47 μl, 0.41 mmol), in toluene (2 ml), was heated in a microwave tube at 170° C. for 20 minutes. The cooled mixture was then concentrated in vacuo and the residue was purified by column chromatography on silica gel, eluting with dichloromethane:methanol:0.88 ammonia, 90:10:1 to 70:30:3, to afford the title compound as a white solid in 76% yield, 214 mg.

¹HNMR(400 MHz, CDCl₃) δ: 3.34(s, 3H), 4.45(s, 2H), 6.82(m, 1H), 7.18(m, 1H), 7.25(s, 1H), 7.35(d, 2H), 7.49(d, 2H), 7.81(s, 1H), 8.09(d, 1H); LRMS APCI m/z 340 [M+H]⁺

All examples described above were tested in the screens described above, and all compounds exhibit a V1a Ki value of 450 nM or less. Examples of specific compounds are illustrated below:

| Example No. | Ki (nM) |
|---|---|
| 1 | 9.69 |
| 6 | 6.58 |
| 8 | 0.88 |
| 10 | 0.47 |

1. A compound of formula (I) 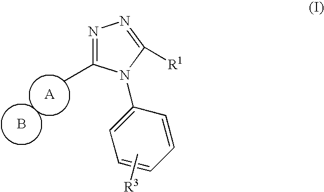
or a pharmaceutically acceptable salt or solvate salt, thereof, wherein
R¹ is [CH₂]ₙ—R²;
R² is H, C₁₋₆ alkyloxy or Het;
n is a number selected from 0 to 6;
Het is an unsaturated heterocycle of 5 or 6 atoms containing one to four heteroatoms selected from O, N, and S;
R³ is halo;
Ring A is a 4 to 7 membered, saturated, partially saturated, or unsaturated heterocycle containing one or more to four heteroatoms selected from O, N, and S;
Ring B is a saturated, partially saturated, or unsaturated heterocycle of from 3 to 8 atoms containing one to four heteroatoms selected from O, N, and S, or Ring B is a saturated or unsaturated carbocyclic ring of from 3 to 8 atoms; and wherein Ring B is optionally fused to an aryl ring and is optionally substituted with one to four groups independently selected from R⁴; and with the proviso that Ring A and Ring B share at least one atom;
R⁴ is oxo, [CH₂]ₘ—R⁵, or CH—R⁶R⁷;
R⁵ is H, OH, C₁₋₆ alkyloxy, COOH, or CONR⁸R⁹;
m is a number selected from 0 or 1; and
R⁶, R⁷, R⁸ and R⁹ are each independently selected from H or C₁₋₆alkyl.

2. A compound according to claim 1, wherein ring A contains I nitrogen atom or a pharmaceutically acceptable salt thereof.

3. A compound according to claim 2, wherein ring A is piperidinyl, pyrrolidinyl or azepinyl or a pharmaceufically acceptable salt thereof.

4. A compound according to claim 1, wherein ring B is phenyl, cyclopentyl, dihydro-furanyl-2-one or furanyl or a pharmaceuticallv acceptable salt thereof.

5. A compound according to claim 1, wherein ring B is fused to a phenyl group or a pharmaceutically acceptable salt thereof.

6. A compound according to claim 1, wherein R⁴ is hydroxymethyl, methoxymethyl or CONH₂ or a pharmaceutically acceptable salt thereof.

7. A compound according to claim 1, which is selected from the group consisting of:
2-[4-(4-Chlorophenyl)-5-(methoxymethyl)-4H-1,2,4-triazol-3-yl]isoindoline;
2-[4-(4-Chlorophenyl)-5-methyl-4H-1,2,4-triazol-3-yl]isoindoline;
2-[4-(4-Chlorophenyl)-5-methyl-4H-1,2,4-triazol-3-yl]-2,3,4,9-tetrabydro-1H-β-carboline;
1′-[4-(4-Chlorophenyl)-5-methyl-4H-1,2,4-triazol-3-yl]-3H-spiro[2-benzofturan-1,4′-piperidin]-3-one;
1′-[4-(4-chlorophenyl)-5-methyl-4H-1,2,4-triazol-3-yl]spiro[isoindole-1,4′-piperidin]-3(2H)-one;
1′-[4-(4-Cblorophenyl)-5-methyl-4H-1,2,4-triazol-3-yl]-3H-spiro[2-benzofuran-1,4′-piperidine];
1′-[4-(4-Chlorophenyl)-5-methyl-4H-1,2,4-triazol-3-yl]-2-methylspiro[isoindole-1,4′-piperidin]-3(2H)-one;
2-[4-(4-Chlorophenyl)-5-(2H-1,2,3-triazol-2-ylmethyl)-4H-1,2,4-triazol-3-yl]isoindoline;
6-[4-(4-Chlorophenyl)-5-(2H-1,2,3-triazol-2-ylmethyl)-4H-1,2,4-triazol-3-yl]-6,7-dihydro-5H-pyrrolo[3,4-b]pyridine;
{2-[4-(4-Chlorophenyl)-5-(2H-1,2,3-triazol-2-ylmethyl)-4H-1,2,4-triazol-3-yl]-2,3-dihydro-1H-isoindol-5-yl}methanol;
1′-[4-(4-chlorophenyl)-5-methyl-4H-1,2,4-triazol-3-yl]-2,3-dihydrospiro[indene-1,4′-piperidine];
{2-[4-(4-Chlorophenyl)-5-methyl-4H-1,2,4-triazol-3-yl]-2,3-dihydro-1H-isoindol-5-yl}methanol;
2-[4-(4-Chlorophenyl)-5-methyl-4H-1,2,4-triazol-3-yl]-5-(methoxymethyl)isoindoline;
2-[4-(4-Chlorophenyl)-5-(2H-1,2,3-triazol-2-ylmethyl)-4H-1,2,4-triazol-3-yl]isoindoline-5-carboxylic acid;
3-[4-(4-Chlorophenyl)-5-methyl-4H-1,2,4-triazol-3-yl]-2,3,4,5-tetrahydro-1H-3-benzazepine;
2-[4-(4-Chloro-phenyl)-5-[1,2,3]triazol-2-ylmethyl-4H-[1,2,4]triazol-3-yl]-quinoxaline;
2-[4-(4-Chloro-phenyl)-5-[1,2,3]triazol-2-ylmethyl-4H-[1,2,4]triazol-3-yl]-[1,6]naphthyridine;
3-[4-(4-Chloro-phenyl)-5-[1,2,3]triazol-2-ylmethyl-4H-[1,2,4]triazol-3-yl]-1H-indazole
5-[4-(4-Chlorophenyl)-5-(2H-1,2,3-triazol-2-ylmethyl)-4H-1,2,4-triazol-3-yl]-1-methyl-1,4,5,6-tetrahydropyrrolo[3,4-c]pyrazole;
6-[4-(4-Chlorophenyl)-5-(2H-1,2,3-triazol-2-ylmethyl)-4H-1,2,4-triazol-3-yl]-6,7-dihydro-5H-pyrrolo[3,4-d]pyrimidine;
7-[4-(4-Chlorophenyl)-5-(2H-1,2,3-triazol-2-ylmethyl)-4H-1,2,4-triazol-3-yl]-5,6,7,8-tetrahydropyrido[3,4-d]pyrimidine;
6-[4-(4-Chlorophenyl)-5-(2H-1,2,3-triazol-2-ylmethyl)-4H-1,2,4-triazol-3-yl]-2-methyl-6,7-dihydro-5H-pyrrolo[3,4-d]pyrimidine;
7-[4-(4-Chlorophenyl)-5-(2H-1,2,3-triazol-2-ylmethyl)-4H-1,2,4-triazol-3-yl]-2-methyl-5,6,7,8-tetrahydropyrido[3,4-d]pyrimidine;
5-[4-(4-Chlorophenyl)-5-(2H-1,2,3-triazol-2-ylmethyl)-4H-1,2,4-triazol-3-yl]-5,6-dihydro-4H-pyrrolo[3,4-c]isoxazole;
5-[4-(4-Chlorophenyl)-5-(2H-1,2,3-triazol-2-ylmethyl)-4H-1,2,4-triazol-3-yl]-2-methyl-4,5,6,7-tetrahydro-2H-pyrazolo[4,3-c]pyridine;
5-[4-(4-Chlorophenyl)-5-(2H-1,2,3-triazol-2-ylmethyl)-4H-1,2,4-triazol-3-yl]-2-isopropyl-4,5,6,7-tetrahydro-2H-pyrazolo[4,3-c]pyridine;
1-[4-(4-Chloro-phenyl)-5-methyl-4H-[1,2,4]triazol-3-yl]-2,3-dihydro-1H-indole-6-carboxylic acid amide;
1-[4-(4-Chloro-phenyl)-5-methyl-4H-[1,2,4]triazol-3-yl]-2,3-dihydro-1H-indole-6-carboxylic acid dimethylamide;
1-[4-(4-Chloro-phenyl)-5-[1,2,3]triazol-2-ylmethyl-4H-[1,2,4]triazol-3-yl]-2,3-dihydro-1H-indole-6-carboxylic acid amide;
1-[4-(4-Chloro-phenyl)-5-[1,2,3]triazol-2-ylmethyl-4H-[1,2,4]triazol-3-yl]-2,3-dihydro-1H-indole-6-carboxylic acid methylamide; and
2-[4-(4-Chlorophenyl)-5-(methoxymethyl)-4H-1,2,4-triazol-3-yl]imidazo[1,2-a]pyridine;
or a pharmaceutically acceptable salt thereof.

8.-9. (canceled)

10. A method of treating anxiety, cardiovascular disease primary dysmenorrhea, secondary dysmenorrhea, endometriosis, emesis, intrauterine growth retardation, inflammation, mittlesmerchz, preclampsia, premature ejaculation, premature labor or Raynaud's disease in a mammal, the method comprising administering to the mammal in need thereof a therapeutically effective amount of a compound according to claim 1 or a pharmaceutically acceptable salt thereof.

11. The method according to claim 10, wherein primary dysmenorrhea or secondary dysmenorrhea is treated.

12.-14. (canceled)

15. A pharmaceutical composition comprising a compound according to claim 1, together with a pharmaceutically acceptable excipient, diluent or carrier.

16. A pharmaceutical composition comprising (A) a compound according to claim 1 and (B) another pharmacologically active ingredient.

17. The pharmaceutical composition according to claim 16, wherein (B) is an oral contraceptive, PDEV inhibitor, COX inhibitor, NO-donor or L-arginine.

18. (canceled)

19. A method of treating primary dysmenorrhea or secondary dysmenorrhea in a mammal, the method comprising administering to a the mammal in need of such treatment a therapeutically effective amount of the pharmaceutical composition according to claim 17.

20. (canceled)
